(19) United States
(12) Patent Application Publication
(10) Pub. No.: US 2004/0253635 A1
(43) Pub. Date: Dec. 16, 2004

(54) Methods of identifying functional analogs of peptide regulators of biological pathways

(76) Inventor: Sylvie Luria, Nes Ziona (IL)

Correspondence Address: Anthony Castorina, G E Ehrlich, Suite 207, 2001 Jefferson Davis Highway, Arlington, VA, 22202, US

(21) Appl. No.: 10/484,440

(22) PCT Filed: Aug. 6, 2002
(86) PCT No.: PCT/IL02/00646

Related U.S. Application Data
(60) Provisional application No. 60/309,778, filed on Aug. 6, 2001.

(51) Int. Cl. G01N 33/53; G01N 33/543

(52) U.S. Cl. 435/7.1; 436/518

(57) ABSTRACT

A method of uncovering a putative functional analog of a peptide regulator of a biological pathway is disclosed. The method comprises: (a) generating a library including molecules representing: (i) constituents of the biological pathway; and/or (ii) portions of said constituents of the biological pathway; (b) contacting said molecules of said library with the peptide regulator to thereby obtain a complex composed of a molecule of said molecules of said library and the peptide regulator; (c) incubating said molecule and the peptide regulator of said complex in the presence of each of a plurality of distinct substances; and (d) identifying a substance of said plurality of distinct substances capable of competing with the peptide regulator for binding of said molecule to thereby uncover the putative functional analog of the peptide regulator of the biological pathway.

### FIELD AND BACKGROUND OF THE INVENTION

The present invention relates to methods of characterizing molecular targets of molecular regulators of biological pathways. More particularly, the present invention relates to methods of characterizing peptide targets of molecular regulators of biological pathways and using such peptide targets and such molecular regulators to uncover putative functional analogs of such molecular regulators having desired physical, chemical, and/or biological characteristics.

Intermolecular or intramolecular interactions are central to the function and regulation of biological processes, such as biochemical events and cellular processes. The vast number of such events, which include processes as diverse as DNA synthesis, transcriptional activation, protein translation, localization and translocation of cellular molecules, molecular secretion, cell cycle control, intermediary metabolism, pathogen invasion, cellular signal transduction, and so on, operate and are regulated via highly specific intermolecular and intramolecular interactions. Such intermolecular interactions often involve formation of molecular complexes which may comprise combinations of various types of molecules, such as proteins, nucleic acids, and carbohydrates.

Regulators capable of activating biological pathways include molecules causing propagation of a cellular event upon their binding to a cognate binding protein. Examples of such positive regulators include hormones, growth factors, antibodies, and peptides. Regulators capable of inhibiting biological pathways include molecules inhibiting propagation of a cellular event upon their binding to a cognate binding protein. Examples of such negative regulators include negative regulators of transcription, such as IkB which inhibits NF-κB, molecules such as IGF-I binding proteins that bind to IGF-I and interfere with its binding to its receptors, molecules acting as silencers of transcription, and enzymes such as kinases or phosphatases that negatively modulate cellular signal transduction.

Consistent with the functional importance of molecular interactions in generating and regulating biological phenotypes, many diseases occur as a consequence of particular alterations or as a consequence of disregulation of such interactions. For example, one of the key events in the pathogenesis of malignant diseases, is the disregulation of biological pathways, such as growth factor receptor activated pathways, for example insulin-like growth factor-1 receptor (IGF-I receptor) activated pathways.

IGF-I receptor signaling: IGF-I receptor is a growth factor involved in biological pathways participating in cell growth, cell differentiation, cell transformation and protection from apoptosis (Butler A A. et al., 1998. Comp Biochem Physiol B Biochem Mol Biol. 121(1):19-26; Clemmons D R., 1992. Growth Regul. 2:80-87). Similarly to other tyrosine kinase growth factor receptors, binding to IGF-I induces receptor autophosphorylation which triggers cellular signal transduction pathways (Kato H. et al., 1993. J Biol Chem. 265:2655-2661; Gronborg M. et al., 1993. J Biol Chem. 258:23435-23440). Various steps of IGF-I receptor signaling have been elucidated. For example, activation of IGF-I receptor induces insulin receptor substrate-1 (IRS-1) and insulin receptor substrate-2 (IRS-2) protein phosphorylation, thereby promoting a mitogenic response. Activation of IGF-I receptor also induces phosphorylation of the SH2 and SH3 domains of proteins of the proto-oncogene CrkII and of CrkL (Koval A P. et al., 1998. J Biol Chem. 273:14780-14787), and activation of phosphatidylinositol-3-kinase (Bruning J C. et al., 1997. Mol Cell Biol. 17:1513-1521). The IGF-I activation cascade culminates in transcription of several genes, including vascular endothelial growth factor (VEGF) and hexokinase II (Sebastian S. and Kenkare U W., 1997. Biochem Biophys Res Commun. 235:389-393; Warren R S. et al., 1996. J Biol Chem. 271:29483-29488; Akagi Y. et al., 1998. Cancer Res. 58:4008-4014). EHD-1 has been shown to interact with the IGF-I receptor to mediate its endocytosis and to control its off pathway (Mintz L. et al., 1999. Genomics 59:66-76; U.S. Pat. application No. 09/026,898).

Thus, a large number of molecular interactions are involved in mediating growth factor receptor activated biological pathways such as IGF-I receptor activated biological pathways.

Numerous diseases, including malignant diseases having significant morbidity and mortality, such non small-cell lung carcinoma, are associated with disregulation of tumor suppressor associated biological pathways. For example, disregulation of biological pathways associated with tumor suppressors such as p53, Rb, or p16 at genetic, epigenetic, or post-translational levels removes important constraints on cell division in malignant diseases such as non small-cell lung cancer, thereby contributing to their pathogenesis.

Non small-cell lung cancer: Non small-cell lung cancer is the dominant histology in lung cancers, being responsible for 75% to 80% of all lung malignancies. Non small-cell lung cancer is the leading cause of cancer deaths in the seven major pharmaceutical markets (the United States, France, Germany, Italy, Spain, the United Kingdom, and Japan). In the United States and Japan, non small-cell lung cancer accounts for more deaths each year than do colorectal cancer, breast cancer, and prostate cancer combined. Approximately 40% to 50% of non small-cell lung cancer patients present with metastatic (Stage IV) disease. Furthermore, because of early hematogenous spread, most patients presenting with earlier-stage disease will eventually develop metastatic disease.

Thus, numerous diseases are associated with disregulation of biological pathways.

There is therefore a vital need for methods of identifying substances capable of regulating biological pathways and being suitable for treating diseases associated with disregulation of such biological pathways.

One strategy for treating diseases associated with disregulation of biological pathways, such as diseases associated with disregulation of growth factor receptor activated biological pathways or diseases associated with disregulation of tumor suppressor associated pathways, is to employ molecules capable of specifically interacting with constituents of such pathways in such as a way as to produce a therapeutic alteration in the regulation of such biological pathways.

Various prior art approaches for identifying substances capable of regulating biological pathways and being suitable for treating diseases associated with disregulation of such biological pathways have been employed.

Such prior art approaches have employed various combinations of libraries of biologically derived or synthetic candidate regulatory molecules, and target molecule-binding assays or functional assays for screening such libraries.

In approaches using target molecule-binding assays for identifying candidate regulatory molecules, libraries of molecules such as peptides, proteins, or small molecule compounds are screened for molecules having the capacity to interact with target molecules which are known and characterized, such as receptors, antibodies or enzymes. Libraries are screened for molecules having the ability, for example, to bind target molecules, to interfere with formation of molecular complexes comprising target molecules, or to interfere with a functionality of the target molecule.

Approaches employing target molecule-binding assays have employed various techniques for identifying molecules capable of interacting target molecules. Such techniques include protein affinity chromatography, affinity blotting, co-immunoprecipitation, molecular cross-linking, solid-phase protein arrays (Zu and Snyder, 2001. Curr Opin Chem Biol. 5:40-45), protein tagging, the yeast two hybrid system, the yeast three-hybrid system, and display technologies.

In approaches employing functional assays for identifying candidate regulatory molecules, specific molecules are selected capable of binding a target molecule, or of interfering with intracellular signaling. In general, cultured cells are treated with libraries of molecules such as peptides, proteins, or small molecule compounds, and specific cellular events are monitored. Compounds that modulate the signal of interest are selected as modulators (inhibitors or activators) of that specific biochemical event or cellular pathway (for review, see Phizicky E M. and Fields S., 1995. Microbiol Rev. 59:94-123).

Approaches employing functional assays have employed various techniques for identifying molecules capable of regulating biological pathways. Such techniques have included the use of reporter genes under the regulatory control of a promoter activated by such biological pathways (Luria S., WO00138569A1).

Various approaches using libraries of synthetic molecules have been employed for identifying candidate regulatory molecules. Such approaches have used combinatorial libraries of molecules such as peptides, nucleic acids (Ellington and Szostak, 1990. Nature 246:818), oligonucleotides, peptoids (Simon et al., 1992. Proc Natl Acad Sci U S A. 89:9367-71), carbohydrates and small organic molecules (Eichler et al., 1995. Med Res Rev. 15:481-96).

Various approaches using libraries of biological molecules have been employed for identifying candidate regulatory molecules. Such approaches have used combinatorial libraries, and libraries of proteins, peptides, nucleic acids, and carbohydrates.

Protein tagging: Protein tagging methods utilize fusion proteins comprising a peptide tag, such as a peptide epitope, an enzymatically active polypeptide, or a fluorescent polypeptide, and a defined protein sequence of interest. Such chimeras are usually generated by recombinant DNA sequences encoding both polypeptides in tandem. Such chimeras can serve, for example, as tools to localize cellular target proteins, or to isolate molecules such as proteins or nucleic acids which interact with the target protein, by using the tag sequence as an indicator of the tagged molecule. For example, proteins of interest conjugated to peptide tags consisting of six histidine amino acids can be visualized when expressed in cells by immuno-staining using anti-tag antibodies. As well, such tags can be used to isolate proteins which specifically form protein complexes the protein of interest via tag-specific affinity column capture or tag-specific immunoprecipitation (Skolnik et al., 1991. Cell 65:83-90). Using the same approach, one can tag transcription factors that interact with other proteins and DNA, and track their cellular localization and activity during cellular pathways. Additionally, proteins can be tagged with fluorescent proteins, enabling localization of the protein in real time in live cells and organisms. Several proteins can be tagged simultaneously using different tags, enabling more complex analysis of molecular interactions.

Yeast two-hybrid system: The yeast two-hybrid system is a useful way to detect proteins that interact with a protein of interest. In general, it is used for initial identification of interacting proteins. The two-hybrid system is a system employing transcriptional activity, typically using lacZ as a reporter gene, as a readout to measure protein-protein interactions. This system takes advantage of the modular nature of many site specific transcriptional activators which consist of a DNA binding domain and a transcriptional activation domain (Chein, C T. et al., 1991. Proc Natl Acad Sci USA. 88:9578-9582; Fields S. and Song O K., 1989. Nature 340:245-246; Fields S. and Sternglanz R., 1994. Trends Genet. 10:286-292). The DNA binding domain serves to target the activator to a specific gene to be expressed, while the activation domain binds molecules of the transcriptional machinery to thereby initiate transcription. The two domains of the transcriptional activator need not be covalently linked but simply brought into proximity to initiate transcription. The two domains of the transcriptional activator can be brought into proximity by a pair of interacting proteins. This is achieved by constructing two hybrids, a first hybrid in which the DNA binding domain of the transcriptional activator fused to a first protein (often termed the “bait”), and a second hybrid in which the transcription activation domain of the transcriptional activator is fused to a second protein (often termed the “prey”). In the activation domain hybrid, a recombinant DNA “library” is usually prepared in which genes for many different proteins are fused to the activation domain. These two-hybrids are over-expressed in a cell containing one or more reporter genes under the control of a cis acting element that is known to be bound by the DNA binding domain. If the first and second proteins interact, the domains of the activator are brought into proximity and the reporter gene is activated. Since the two-hybrid system involves the utilization of nucleus functioning transcriptional activator, this system is limited to interactions which can occur in the nucleus, thus preventing its use with certain extra-cellular proteins. Initially, the DNA binding and active domains of the yeast protein GAL4 were employed, whereas subsequent studies have employed the DNA binding domain of the E. coli protein LexA.

Typically, combinatorial expression libraries employed to generate activation domain hybrids contain greater than 106 different different clones, a sufficiently high number so as to generally include a few clones able to interact with the bait. These few can then be recognized by their ability to turn on the reporter gene.

Numerous variations on the two-hybrid system have been employed, such as the reverse two-hybrid system described in U.S. Pat. No. 5,965,368 to Vidal et al.

Yeast three-hybrid system: The three-hybrid system can be used to analyze interactions between three distinct components. This system is typically used to detect and analyze RNA-protein interactions in which the binding of bifunctional RNA to each of two hybrid proteins activates transcription of a reporter gene in-vivo. This binding relies on the physical properties of the RNA and proteins and not on their natural biological activities (SenGupta D J. et al., 1996. Proc Natl Acad Sci USA. 93:8496-8501). In this system, the third protein or RNA can participate in the interaction in several ways, for example as a “bridge” interacting with two proteins that do not directly interact with each other, by stabilizing a weak interaction between two proteins, or by inhibiting the two-hybrid interaction. In this way, proteins peptides or small chemical compounds can be isolated that inhibit the interaction between two proteins.

Cellular approaches to detect and isolate polypeptides from a library of polypeptides, that modulate protein interactions and regulate cellular transduction pathways have been described in patent application # PCT/IL00/00680 (1999). These methods use transcription libraries of tagged peptides and screening of modulators of cellular pathways through cellular reporter systems.

Additional hybrid methods to study protein. interactions with DNA, RNA and small molecules include the analysis of protein interactions in bacteria, bacteria n-hybrid systems, to examine interactions (Hu, 2001. Trends in Microbiology 9:219-222).

Display technologies: The use of surface display vectors for displaying polypeptides on the surface of phages, bacteria, animal viruses or eukaryotic cells, combined with in-vitro selection technologies, enables the manipulation and screening of combinatorial libraries of various types of molecules, such as receptor ligands, enzymes, antibodies, nucleic acids and peptides, for members of such libraries having selected phenotypes (Benhare I, 2001. Biotechnology Advances 19:1-33; Griffiths AD. et al., 1998. Curr Opin Biotechnology 9:102-108; Smith GP. et al., 1985. Science 228:1315-1317).

Phage and viral display technologies are based on expressing recombinant proteins, such as variable regions of antibodies, or peptides fused to phage or viral coat proteins. Bacterial and cellular display technologies are based on expressing recombinant proteins or peptides fused to sorting signals that direct them to the cell surface. In both systems, the genetic information encoding for the displayed molecules is linked to its product via the displaying particle, thus enabling cloning of nucleic acid sequences encoding molecules having selected characteristics.

Combinatorial peptide and protein libraries can be expressed in such systems to study protein interactions and ligand binding. Among the various existant display technologies, M13 phage display is becoming of rapidly increasing importance in immunology, cell biology, protein biochemistry, protein engineering, gene transfer and pharmacology. It has been used to create combinatorial libraries of peptides, protein domains and proteins, such as single-chain Fv (scFv) and Fab,. to generate antibodies having novel binding specificities, to identify target molecule ligands, such as peptide ligands or antibodies, or to identify antibody epitopes.

Phage particles consist of a nucleic acid molecule surrounded by a proteinaceous coat, which enables the phage to interact with, and infect, host bacteria. Filamentous phages, such as M13, can express a fusion protein bearing a foreign peptide on the coat surface by infecting a bacterial host such as E. coli (Smith G P., 1985. Science 228:1315-1317). DNA sequences coding for protein or peptide of interest are translationally fused to the 5′ end of the gene encoding one of the phage coat proteins (e.g., Vp3 or Vp8 in M13). If the translational fusion does not interfere with the life cycle of the phage, the modified phage particle will express a chimeric coat protein which displays the foreign peptide or protein of interest. Phage particles “displaying” the foreign peptide or protein on their surface can be selected by affinity purification. Phage display libraries can be prepared by constructing a collection of phage particles each capable of displaying a different foreign peptide or protein. Different types of proteins, such as secreted, as well as cytoplasmic and nuclear proteins, have been displayed successfully on phages, displayed on bacteria and on eukaryotic cells (Crameri R and Suter M, 1993, Gene 137:69-75; George R et al., 2000, Drug Discovery Technologies, 4:145-156)

Random peptide phage display libraries have proven to be a useful tool to identify the protein constituents of various protein-protein interaction reactions (Parmley S F. and Smith G P., 1989. Adv Exp Med Biol. 251:215-218; Scott J K. and Smith G P. 1990. Science 249:386-390; Winter, J. 1994. Drug and Dev Result. 33:71-89). Such libraries have also been used to define epitopes of monoclonal and polyclonal antibodies and to define the specificity of extracellular and cytosolic receptors (Devlin et al., 1990. Science 249:404-406; Doorbar J. and Winter G., 1994. J Mol Biol. 244:361-369; Kay B K. 1995. Perp Drug Disc. 2:251-268).

Combinatorial libraries: In combinatorial libraries, chemical building blocks are randomly combined into a large number of different compounds, which are then simultaneously screened for binding (or other) activity against one or more targets. Libraries containing up to millions of random peptides have been prepared by chemical synthesis (Houghten et al., 1991. Nature 354:84-6) or by gene expression (Marks et al., 1991. J Mol Biol. 222:581-97).

Such combinatorial libraries have been generated displaying molecules on chromatographic supports (Lam et al., 1991. Nature 354:82-4), inside bacterial cells (Colas et al., 1996. Nature, 380:548-550), on bacterial pili (Lu, Bio/Technology, 13:366-372 (1990)), on phages (Smith, 1985. Science 228:1315-7), and have been used to screen for molecules binding to a variety of targets, including antibodies (Valadon et al., 1996. J Mol Biol. 261:11-22), cellular proteins (Schmitz et al., 1996. J Mol Biol. 260:664-677), viral proteins (Hong and Boulanger, 1995. Embo J. 14:4714-4727), bacterial proteins (Jacobsson and Frykberg, 1995. Biotechniques 18:878-885), nucleic acids (Cheng et al., 1996. Gene 171:1-8), and plastic (Siani et al., 1994. J Chem Inf Comput Sci. 34:588-593).

Combinatorial libraries of proteins (Ladner, U.S. Pat. No. 4,664,989), peptoids (Simon et al., 1992. Proc Natl Acad Sci U S A. 89:9367-71), nucleic acids (Ellington and Szostak, 1990. Nature, 246:818), carbohydrates, and small organic molecules (Eichler et al., 1995. Med Res Rev. 15:481-96) have also been prepared or suggested for drug screening purposes.

For various reasons, small molecules are optimal candidates for drug development and pharmaceutical use, therefore much work on combinatorial libraries has involved small molecules. The techniques of combinatorial chemistry have been recognized as the most efficient means for identifying small molecules having the capacity to act on potential targets in-vitro. At present, small molecule combinatorial chemistry involves the synthesis of either pooled or discrete molecules presenting varying arrays of functionality on a common scaffold. These compounds are grouped in libraries that are screened against target molecules of interest, either for binding or for regulation of a biological activity.

The typical way of screening chemical compound libraries starts with the identification of a target molecule, say an enzyme, a fragment of DNA, an antibody or a receptor. An assay is developed for each target to select for small molecule, or any other ligand that interacts with the target molecule, or inhibits or activates a biological pathway (Christensen et al., 2001, DDT 2001, 6:721-727; Lenz et al., 2000. DDT 5:145-152; Herzberg et al., 2000. Curr Opin Chem Biol. 4:445-451).

However, all of the aforementioned approaches for identifying candidate regulators of biological pathways being suitable for treating diseases associated with disregulation of such biological pathways suffer from significant disadvantages.

Approaches using binding assays and/or screens of synthetic molecule libraries to identify candidate regulators of biological pathways are highly inefficient since candidate regulators of biological pathways identified via such approaches inherently have a suboptimal probability of being capable of regulating such biological pathways relative to approaches using functional assays, and/or screens of biological molecule libraries, respectively. Whereas only a small quantity of a molecule associated with a biological pathway, may be required to modulate a particular cellular response, screening libraries of synthetic molecules for molecules having such capacity requires very large-scale screening and the ability to achieve high concentrations of the chemical agent. Furthermore, approaches using binding assays rely on the assumption the molecule of a biological pathway for which a ligand is sought can be used targeted by a ligand so as to regulate the biological pathway.

Approaches utilizing functional assays and/or screens of biological molecule libraries to identify candidate regulators of biological pathways are optimal for selecting polypeptide candidate regulators of biological pathways. However, polypeptides are highly unsuitable as pharmacological agents, being costly to synthesize, unstable under physiological conditions, and having suboptimal biodistribution capacity, for example due to their being highly inefficient at crossing cell membranes.

Thus, all prior art approaches have failed to provide an adequate solution for efficiently uncovering substances capable of regulating biological pathways and being suitable for treating diseases associated with disregulation of such biological pathways.

There is thus a widely recognized need for, and it would be highly advantageous to have, methods of uncovering substances capable of regulating biological pathways and being suitable for treating diseases associated with disregulation of such biological pathways devoid of the above limitation.

### SUMMARY OF THE INVENTION

According to one aspect of the present invention there is provided an expression construct system comprising a plurality of expression constructs being for phage display expression of polypeptides, each of the expression constructs having a unique polylinker sequence flanked by: (a) a first polynucleotide region encoding a phage leader sequence positioned upstream of the polylinker; and (b) a second polynucleotide region encoding a chimeric polypeptide including a tag sequence fused to a phage coat protein; wherein each unique polylinker is designed to enable cloning of a desired polynucleotide in a unique reading frame combination with respect to the leader sequence and the chimeric polypeptide, such that phage particles expressing the desired polynucleotide cloned in frame to the leader sequence and the chimeric polypeptide can be identified and optionally isolated from a phage particle population transformed with the plurality of expression constructs harboring the desired polynucleotide.

According to further features in preferred embodiments of the invention described below, the phage leader sequence is a gene 3 leader sequence.

According to still further features in preferred embodiments, the tag sequence is selected from the group consisting of a fluorescent tag, an enzyme tag, an epitope tag, and an affinity tag.

According to still further features in preferred embodiments, the phage coat protein is phage coat protein III.

According to still further features in preferred embodiments, the phage particles are M13 phage particles.

According to still further features in preferred embodiments, the desired polynucleotide is a cDNA encoding at least a portion of a constituent of a biological pathway.

According to another aspect of the present invention there is provided a method of uncovering a putative functional analog of a peptide regulator of a biological pathway, the method comprising: (a) generating a library including molecules representing: (i) constituents of the biological pathway; and/or (ii) portions of the constituents of the biological pathway; (b) contacting the molecules of the library with the peptide regulator to thereby obtain a complex composed of a molecule of the molecules of the library and the peptide regulator; (c) incubating the molecule and the peptide regulator of the complex in the presence of each of a plurality of distinct substances; and (d) identifying a substance of the plurality of distinct substances capable of competing with the peptide regulator for binding of the molecule to thereby uncover the putative functional analog of the peptide regulator of the biological pathway.

According to further features in preferred embodiments of the invention described below, the peptide regulator comprises a detectable tag, and step (d) is effected by detecting dissociation of the detectable tag from the molecule of the molecules of the library.

According to still further features in preferred embodiments, the plurality of distinct substances is a plurality of molecules each having a lower molecular weight than that of the peptide regulator.

According to still further features in preferred embodiments, the plurality of distinct substances is a plurality of molecules each having a volume smaller than that of the peptide regulator.

According to yet another aspect of the present invention there is provided a method of uncovering a putative functional analog of a molecular regulator of a biological pathway, the method comprising: (a) generating a library including molecules representing: (i) constituents of the biological pathway; and/or (ii) portions of the constituents of the biological pathway; (b) contacting the molecules of the library with the molecular regulator to thereby obtain a complex composed of a molecule of the molecules of the library and the molecular regulator; (c) incubating the molecule and the molecular regulator of the complex in the presence of each of a plurality of distinct substances; and (d) identifying a substance of the plurality of distinct substances capable of competing with the molecular regulator for binding of the molecule to thereby uncover the putative functional analog of the molecular regulator of the biological pathway.

According to further features in preferred embodiments of the invention described below, the molecular regulator is a molecule selected from the group consisting of a polypeptide, a polynucleotide, a carbohydrate, a biological polymer, and an inorganic molecule.

According to still further features in preferred embodiments, the molecular regulator comprises a molecule selected from the group consisting of a polypeptide, a polynucleotide, a carbohydrate, a biological polymer, and an inorganic molecule.

According to still further features in preferred embodiments, the molecular regulator comprises a detectable tag, and step (d) is effected by detecting dissociation of the detectable tag from the molecule of the molecules of the library.

According to still further features in preferred embodiments, the molecules of the library comprise a detectable tag, and step (d) is effected by detecting dissociation of the detectable tag from the molecular regulator of the complex.

According to still further features in preferred embodiments, the each of a plurality of distinct substances comprises a detectable tag, and step (d) is effected by detecting association of the detectable tag with the molecule of the molecules of the library.

According to still further features in preferred embodiments, the detectable tag is selected from the group consisting of a fluorescent tag, an enzyme tag, an epitope tag, and an affinity tag.

According to still further features in preferred embodiments, the fluorescent tag is selected from the group consisting of green fluorescent protein, blue fluorescent protein, FITC and rhodamine.

According to still further features in preferred embodiments, the enzyme tag is selected from the group consisting of beta-galactosidase, horseradish peroxidase and alkaline phosphatase.

According to still further features in preferred embodiments, the affinity tag is selected from the group consisting of a poly-histidine tag, a cellulose binding domain, biotin, avidin, streptavidin, and a DNA-binding domain.

According to still further features in preferred embodiments, the plurality of distinct substances is a plurality of non polypeptide molecules.

According to still further features in preferred embodiments, the plurality of distinct substances is a plurality of molecules each having a lower molecular weight than that of the molecular regulator.

According to still further features in preferred embodiments, the plurality of distinct substances is a plurality of molecules each having a volume smaller than that of the molecular regulator.

According to still another aspect of the present invention there is provided a method of characterizing a molecular target of a molecular regulator of a biological pathway, the method comprising: (a) generating a library including molecules representing: (i) constituents of the biological pathway; and/or (ii) portions of the constituents of the biological pathway; and (b) screening the molecules of the library for a molecule capable of specifically binding the molecular regulator of the biological pathway, thereby characterizing the molecular target of the molecular regulator.

According to further features in preferred embodiments of the invention described below, screening the library comprises: (i) attaching the molecular regulator to a substrate; (ii) exposing the molecular regulator to the molecules of the library, to thereby obtain complexes each composed of the molecular regulator and a molecule of the molecules; and (iii) isolating the complexes.

According to still further features in preferred embodiments, the molecular regulator is a polynucleotide.

According to still further features in preferred embodiments, the polynucleotide includes a gene regulatory element.

According to still further features in preferred embodiments, the gene regulatory element is a promoter.

According to still further features in preferred embodiments, said promoter is a vascular endothelial growth factor promoter or an apoptotic protease activating factor-1 promoter.

According to an additional aspect of the present invention there is provided a method of characterizing a molecular target of a peptide regulator of a biological pathway, the method comprising: (a) generating a library including molecules representing: (i) constituents of the biological pathway; and/or (ii) portions of the constituents of the biological pathway; and (b) screening the molecules of the library for a molecule capable of specifically binding the peptide regulator of the biological pathway, thereby characterizing the molecular target of the peptide regulator.

According to further features in preferred embodiments of the invention described below, screening the library comprises: (i) attaching the peptide regulator to a substrate; (ii) exposing the peptide regulator to the molecules of the library, to thereby obtain complexes each composed of the peptide regulator and a molecule of the molecules; and (iii) isolating the complexes.

According to still further features in preferred embodiments, the method of characterizing a molecular target, further comprises identifying the molecule of the complexes isolated in step (iii).

According to still further features in preferred embodiments, the library is a display library.

According to still further features in preferred embodiments, the display library is a cDNA display library.

According to still further features in preferred embodiments, step (a) comprises fragmenting a pool of polynucleotides by treatment with DNase, or by treatment with a restriction enzyme cleaving at a recognition sequence comprising a number of base pairs numbering less than a range selected from 3 to 7 base pairs, thereby generating a population of polynucleotides encoding the molecules of the library.

According to still further features in preferred embodiments, the restriction enzyme is Rsa I or EcoR V.

According to still further features in preferred embodiments, the display library is a phage display library.

According to still further features in preferred embodiments, the phage display library is a phage display library of polypeptides.

According to still further features in preferred embodiments, the polypeptides are composed of a number of amino acid residues less than a range selected from 3 to 34 amino acid residues.

According to still further features in preferred embodiments, the polypeptides comprise at least portions of signaling intermediates of the biological pathway.

According to still further features in preferred embodiments, the library is prepared from cells containing the constituents of the biological pathway.

According to still further features in preferred embodiments, the molecules are polypeptides and the cells are induced to express the polypeptides.

According to still further features in preferred embodiments, the biological pathway is associated with regulation of apoptosis and the inducing is effected by treatment with Taxol and/or doxorubicin.

According to still further features in preferred embodiments, the biological pathway is an IGF-I receptor activated biological pathway and the inducing is effected by treatment with IGF.

According to still further features in preferred embodiments, the library is a cDNA subtraction library constructed to encode polypeptides unique to cells expressing the biological pathway.

According to still further features in preferred embodiments, the library is a cDNA subtraction library constructed to encode polypeptides not present in cells expressing the biological pathway.

According to still further features in preferred embodiments, the cDNA subtraction library is derived from a subtraction between a cDNA library generated from cells of a tissue type having a normal phenotype and a cDNA library generated from cells of the tissue type having an abnormal phenotype.

According to still further features in preferred embodiments, the tissue type is pulmonary.

According to still further features in preferred embodiments, the abnormal phenotype is a cancerous phenotype or a transformed phenotype.

According to still further features in preferred embodiments, the molecules of the library are signaling intermediates of the biological pathway.

According to still further features in preferred embodiments, the signaling intermediates are selected from the group consisting of IRS-1, EHD-1, IGF-I receptor, p53, a vascular growth factor promoter, and an apoptotic protease activating factor-1 promoter.

According to still further features in preferred embodiments, the molecules of the library include polypeptides and/or polynucleotides.

According to still further features in preferred embodiments, the polynucleotides include gene regulatory elements.

According to still further features in preferred embodiments, the gene regulatory elements include promoters.

According to still further features in preferred embodiments, the promoters include vascular endothelial growth factor promoters or apoptotic protease activating factor-1 promoters.

According to still further features in preferred embodiments, the biological pathway is associated with an abnormal cellular phenotype.

According to still further features in preferred embodiments, the abnormal cellular phenotype is a cancerous phenotype and/or an apoptosis resistant phenotype.

According to still further features in preferred embodiments, the biological pathway is an IGF-I receptor activated biological pathway.

According to still further features in preferred embodiments, the library is prepared from cells selected from the group consisting of NIH 3T3 cells expressing IGF-I receptor, breast cancer cells, placental cells, NIH L1 cells, and adipocytes.

According to still further features in preferred embodiments, the breast cancer cells are primary breast cancer cells or cells of a breast cancer cell line.

According to still further features in preferred embodiments, the breast cancer cell line is T47D or MCF7.

According to still further features in preferred embodiments, the biological pathway is a biological pathway associated with regulation of apoptosis.

According to still further features in preferred embodiments, the regulation of apoptosis is activation of apoptosis or inhibition of apoptosis.

According to still further features in preferred embodiments, the library is prepared from lung cancer cells.

According to still further features in preferred embodiments, the lung cancer cells are primary cancer cells or cells of a lung cancer cell line.

According to still further features in preferred embodiments, the lung cancer cells are non small-cell lung cancer cells.

According to still further features in preferred embodiments, the cancer cell line is selected from the group consisting of H1299, H522, and H23.

According to still further features in preferred embodiments, the biological pathway is a bacterial biological pathway.

According to still further features in preferred embodiments, the bacterial biological pathway is a Staphylococcus aureus biological pathway.

The present invention successfully addresses the shortcomings of the presently known configurations by providing methodology suitable for identifying targets of regulators of biological pathways and analogs of such regulators.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flow chart depicting a protocol for isolation of peptides capable of regulating IGF-I receptor induced signaling pathways. NIH-3T3 reporter cells expressing a CD4 reporter under the regulatory control of an IGF-I receptor signaling responsive promoter vascular endothelial growth factor (VEGF) are transfected to express peptide library LCL. Transfectants are either treated with IGF-I or not treated. After 18 hours, cells are collected and separated according to positive or negative CD4 expression. Activator peptides are selected from the CD4 positive non-IGF-I treated population, and inhibitor peptides are selected from the CD4 negative IGF-I treated population. Sequences encoding the regulatory peptides are then cloned by PCR from cells displaying such peptide regulation.

FIG. 2 is a flow chart depicting a protocol for isolation of apoptosis inducing peptides. Cells of the human non-small cell lung cancer line H1299 or of the human lung cancer line H522 are transfected to express combinatorial peptides libraries, and after 48 hours, cells are analyzed for expression of the apoptosis marker annexin, and separated according to positive or negative annexin expression. Sequences encoding the regulatory peptides are then cloned by PCR from annexin positive cells. The screening process is performed three times, and individual candidate regulatory peptides are analyzed for their capacity to induce apoptosis.

FIGS. 3a-j are diagrams depicting phagemid vector pCC11 (FIG. 3a) and its set of polylinker adaptors (FIGS. 3bj) utilized to generate a set of vectors used to generate cDNA phage display libraries. The vector backbone is based on a modified pCANTAB5E phagemid (Pharmacia, Uppsala, Sweden) missing the 195 N-terminal codons of the phage pIII gene. The polylinker adaptors are designed to generate nine different vectors for cloning blunt-ended cDNA inserts into the EcoR V site of the polylinker in all possible combinations of reading frames with respect to both the upstream leader sequence and the downstream detectable tag-coat protein III-encoding sequence. This enables one of the nine different vectors to express the cDNA, tag, protein III sequences in frame with the leader sequence, so as to generate a chimeric polypeptide comprising, from the N-terminus to the C-terminus, the cDNA-encoded polypeptide, the tag and protein III. The polylinker adaptors are cloned into the NcoI-NotI sites of pCC11, and the BamH I and EcoR V restriction sites (underlined) of the polylinker adaptors are used for cloning of cDNA fragments.

FIG. 4a is a photograph depicting a coomassie blue stained polyacrylamide gel electrophoretic analysis of a purified chimeric protein comprising the polypeptide regulator EHD-1 a histidine-tag, biotin.

FIG. 4b is a schematic diagram depicting a method for identification and isolation of cellular protein targets of a polypeptide regulator (EHD-1) of a signaling pathway (IGF-I receptor-activated). A purified chimeric protein comprising EHD-1, a histidine-tag, and biotin, is mixed with a cDNA phage display library displaying a chimeric protein comprising the C-terminal domain of phage coat protein III, an affinity tag, and cDNA-encoded sequences derived from cells displaying signaling pathways activated by IGF-I receptor. Specifically interacting phage-regulator molecule (EHD-1) complexes are isolated by affinity separation using a substrate to which a ligand of the affinity tag is conjugated. Individual phages are recovered, propagated and cloned by infection of bacteria therewith, and their cDNA inserts are sequenced to identify and isolate protein targets of the polypeptide regulator.

FIGS. 5a-b are autoradiographs depicting binding of selected phages displaying cellular polypeptides to the polypeptide regulator EHD-1. Different cloned phages selected interacting with EHD-1 displaying cDNA of the human breast cancer cell line T47D were spotted onto nitrocellulose membranes (all non-control grid units in both FIGS. 5a and 5b). In FIG. 5a, controls were spotted with empty phage and EHD-1, or not spotted, and in FIG. 5b, duplicate controls were spotted with EHD-1 or anti-EHD-1 antibody. Spotted membranes were reacted with a chimera comprising EHD-1 fused to a His tag, and the membrane was developed with anti His tag antibodies conjugated to HRP. Positive scoring samples are circled.

FIG. 6 is a schematic diagram depicting a protocol for identification and isolation of cellular polypeptides capable of regulating transcription of genes. A biotinylated promoter is mixed with a cDNA phage display library derived from cells displaying a transduction pathway leading to activation of the gene regulated by the promoter. The cDNA phage displays a chimeric protein containing the C-terminal of phage protein III, an affinity tag, and cDNA-encoded cellular polypeptides. Phages specifically binding the target are isolated by affinity separation using a substrate to which a ligand of the affinity tag is conjugated. Individual phages are then cloned, propagated and their displayed cDNA is sequenced.

FIG. 7 is a schematic diagram depicting a high-throughput protocol for identification of lead functional analogs of regulatory molecules. Complexes composed of tagged regulator molecules and phages displaying a cellular protein specifically bound by the regulator molecule are substrate immobilized on multi-well plate surfaces to which a ligand of the phage has been conjugated. To each well a different compound is incubated with the complexes. After several washes, the presence of the tagged regulator molecules is monitored using HRP-conjugated anti-tag antibodies. Displacement of regulator molecules from complexes is detectable as a reduction in HRP activity. Compounds causing such displacement are selected as functional analogs of regulatory molecules.

FIG. 8 is a schematic diagram depicting a protocol used for identification of lead functional analogs of molecular regulators. Detection tag-conjugated lead regulator peptides of signaling pathways and cDNA phages displaying cellular ligands of such lead regulator peptides are mixed so as to form complexes therebetween. The capacity of compounds to inhibit association of lead peptide regulators in the complexes is measured by adding such compounds to the complexes and monitoring release of the detection tag from the phage.

### DESCRIPTION OF THE PREFERRED EMBODIMENTS

The present invention is of methods of characterizing molecular targets of molecular regulators of biological pathways, expression construct systems used for such characterization, and methods of using such molecular targets and such molecular regulators to uncover putative functional analogs of molecular regulators. Specifically, the present invention utilizes molecule libraries of enriched for constituents of biological pathways to identify specific targets of peptide regulators of such biological pathways and to uncover putative functional analogs of the peptide regulators.

As such the present invention represents an improvement over prior art methods of selecting polypeptide targets of molecular regulators of biological pathways since such prior art methods do not utilize libraries which are enriched for potential targets of peptide regulators, and hence are far less efficient at such selection. Since the present invention provides polypeptide targets of molecular regulators of signaling pathways which can be used, for example, as reagents in binding competition assays, the present invention can further be utilized to uncover substances having the same binding specificities and/or regulatory capacities as such polypeptide targets. In particular the present invention can be used to uncover substances, such as non-polypeptidic substances, further having physical, chemical and/or biological characteristics required, for example, for optimal pharmacological activity, or for optimal drug development, which characteristics not exhibited by polypeptide regulators. Thus, the present invention is superior to prior art methods of uncovering targets of peptide regulators or putative functional analogs thereof.

The principles and operation of the present invention may be better understood with reference to the drawings and accompanying descriptions.

Before explaining at least one embodiment of the invention in detail, it is to be understood that the invention is not limited in its application to the details of construction and the arrangement of the components exemplified in the Examples. The invention is capable of other embodiments or of being practiced or carried out in various ways. Also, it is to be understood that the phraseology and terminology employed herein is for the purpose of description and should not be regarded as limiting.

Characterization of molecular regulators of biological pathways and their targets can potentially be utilized to treat diseases associated with such biological pathways.

Various prior art methods of generating and screening libraries for selecting molecules having the capacity to specifically bind constituents of biological pathways, and of using such molecules to uncover lead compounds for regulating such biological pathways have been described by the prior art.

For example, various libraries, such as display libraries, notably phage-display libraries, representing polypeptides derived from large, non-specific pools of cDNA have been generated and screened for polypeptides capable of binding constituents of biological pathways, and such polypeptides have been used in assays attempting to uncover lead compounds for regulating such biological pathways.

However, such prior art methods are inherently limited. Prior art approaches screen expressible cDNA libraries for polypeptide targets capable of specifically binding a ligand such as a short peptide. Once a ligand-target is identified, further characterization of the polypeptide target is required in order to determine the involvement of such a target in a biological pathway. Once a target which is a constituent of a biological pathway is identified, its respective ligand must be tested for its ability to regulate the biological pathway.

Thus, prior art approaches require high throughput screening in order to identify ligand-target complexes, intensive biochemical screening in order to identify targets that are constituents of biological pathways and functional screening in order to determine the effect of a ligand on a specific biological pathway.

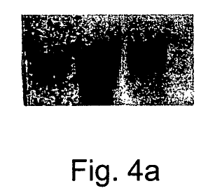

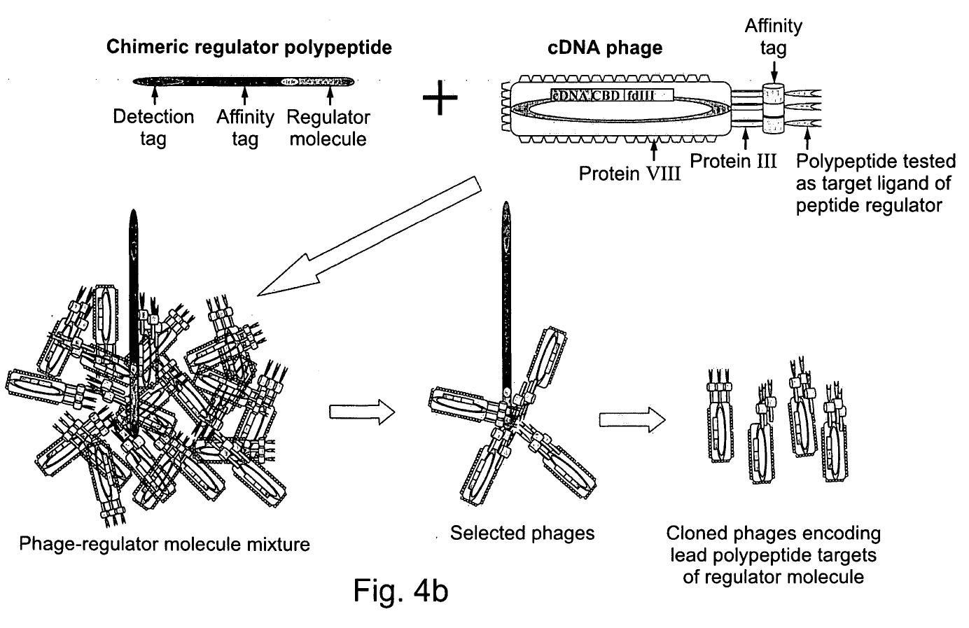

As is clearly illustrated by FIGS. 4a-b, the present invention substantially simplifies the screening process and as such it provides considerable advantages over prior art approaches in both efficiency and accuracy.

The present invention screens a molecule library which includes constituents of a biological pathway against a previously characterized molecular regulator (e.g., peptide regulator) of the biological pathway in order to uncover specific targets of the molecular regulator.

Thus, the present invention forgoes the need for large scale screening approaches and functional assays, thereby substantially simplifying the screening process.

Thus, according to one aspect of the present invention, there is provided a method of characterizing a molecular target of a molecular regulator of a biological pathway.

The method is effected by generating a library including molecules representing constituents of the biological pathway and/or portions of such constituents, and screening the molecules of the library for a molecule capable of specifically binding the molecular regulator of the biological pathway. A molecule or molecules of the library which specifically bind the peptide regulator are further characterized in order to identify the molecular target or targets of the molecular regulator.

As used herein, the terms “constituent of a biological pathway” and “signaling intermediate” are used interchangeably.

As used herein, the phrase “biological pathway” encompasses signaling pathways, transduction pathways, transduction cascades, and the like.

Several types of libraries, can be utilized by the present invention, including, for example, libraries based on cellular expression/presentation of molecules including, for example, eukaryotic cell libraries, prokaryotic cell libraries, viral/phage libraries; and libraries based on non-cellular presentation of molecules, such as, but not limited to, microarray chip libraries, micelle libraries, lipid vesicle libraries, emulsion droplet libraries, and liposome libraries.

Ample guidance regarding the construction and use of various types of libraries is available in the literature of the art (see, for example; Benhare I, 2001. Biotechnology Advances 19:1-33; Griffiths A D. et al., 1998. Curr Opin Biotechnology 9:102-108; Smith G P. et al., 1985. Science 228:1315-1317).

Although such libraries are typically utilized to express and/or present polypeptides, libraries of non-expressible polynucleotides (e.g., promoter sequences) or mixed libraries including non-expressible polynucleotides and polypeptides are also envisioned by the present invention, as well as, libraries of carbohydrates (e.g., polysaccharides). Libraries of non-expressible polynucleotides and polypeptides or carbohydrates are preferably constructed on a substrate such as an array and screened as is further described hereinbelow. Non-expressible polynucleotide libraries can be used to characterize, for example, promoter targets of regulator molecules such as, for example, transcription factor molecular regulators of the biological pathway.

Different types of libraries may be used depending on the nature of the library constituents. For example, the three-dimensional conformation, or the glycosylation of a constituent of a signaling pathway may differ depending on whether the constituent is displayed via eukaryotic cells or prokaryotic cells.

The method of the present invention can be used to characterize a molecular target of a molecular regulator of any desired biological pathway of any prokaryotic or eukaryotic organism. Examples of pathways include an apoptosis pathway a tumorigenesis pathway and the like.

Various type of molecular regulators of biological pathways can be used to screen respective biological pathway libraries.

Examples include, but are not limited to, polypeptides (peptides), polynucleotides, carbohydrates, biological polymers, and inorganic molecules.

Various screening approaches can be used to identify and isolate the target biological pathway constituent which specifically binds with the molecular regulator.

For example, approaches using regulators conjugated to magnetic beads or ligands such as biotin can be utilized to capture and isolate the target constituent. In the case of microarrays, standard scanning methods detecting fluorophore conjugated captured molecules can be used (further detail provided hereinbelow and in the Examples section which follows).

Once isolated, the target constituent can be analyzed using a variety of molecular and biochemical approaches in order to obtain data relating to the molecular target such as, for example, its amino acid residue sequence in the case of polypeptide targets, its nucleic acid sequence in the case of a polynucleotide target, its molecular weight, its binding affinity to another molecule, or a biological function thereof, such as a regulatory function thereof.

As mentioned hereinabove, the library generated and utilized by the present invention can include polypeptides and/or polynucleotides.

Preferably, the library is a polypeptide library which can include polypeptides as large as 50-500 amino acids or smaller than 100 amino acid residues.

Most preferably, the polypeptides of the library are peptides composed of 8 to 66 amino acid residues, more preferably of 12 to 59 amino acid residues, and most preferably of 17 to 33 amino acid residues.

Polypeptide libraries which are expressed from polynucleotides is preferred. Such polynucleotide-encoded polypeptide libraries (e.g., phage display libraries) are advantageous since such libraries greatly facilitate the recovery of nucleic acid sequences encoding displayed molecules cloned in selected elements of the library, and hence the characterization of molecules displayed by such selected elements. Furthermore, elements selected from libraries generated from reproductive elements can be conveniently propagated via the natural reproductive capacity of such elements, for example as described in the Examples section which follows.

Generating polypeptide libraries expressed from polynucleotides is preferably effected by generating a pool of polynucleotides comprising nucleic acid sequences encoding the constituents of the biological pathways, or portions thereof, cloning the pool of polynucleotides in suitable constructs, and expressing such constructs in cells of the library.

The pool of polynucleotides is preferably generated from cells expressing the biological pathway, and hence containing constituents of the biological pathway. Generating the pool of polynucleotides from cells containing constituents of the biological pathway is advantageous since it increases the probability that the pool will encode molecular targets of the regulator molecule which, in turn, increases the probability that an element selected from a library generated from such a polynucleotide pool will display such a molecular target.

Several additional approaches can also be used to further increase the proportion of the biological pathway constituents in the library. For example, cells expressing the biological pathway can be induced to overexpress constituents of the biological pathway and RNA isolated from such cells can then be isolated and used as a template to prepare cDNA. In addition, subtraction libraries of mRNA derived from cells expressing the biological pathway as opposed to cells not expressing the biological pathway can be generated as well as and used as a template for cDNA synthesis.

Various types of polynucleotide pools can be used to generate the library, depending on the application.

Although several type of libraries can be utilized by the present invention including genomic DNA or total RNA libraries, a cDNA library is presently preferred. The use of cDNA libraries is preferable over that of other types of libraries since cDNA libraries are restricted to nucleic acid sequences encoding spliced and expressed molecules, as opposed, for example, to genomic DNA libraries which comprise non-expressed DNA, and non-spliced DNA in the case of eukaryotic DNA, and hence which comprise a large fraction of elements which do not display any molecule, or which display unspliced sequences, and which are thus less efficient for generating libraries from which to select elements displaying molecules having desired properties.

Pools of cDNA can be generated from cells using any one of the numerous standard techniques known in the art. Preferably, pools of cDNA are generated via RT-PCR based methods, as described in the Examples section below.

Generating the library preferably comprises fragmenting the pool of polynucleotides by treatment with DNase, or by treatment with a restriction enzyme cleaving at a recognition sequence comprising a number of base pairs numbering less than a range selected from 3 to 7 base pairs, thereby generating a population of polynucleotides encoding polypeptide fragments of the library.

Preferably, the DNase is DNase I, and the treatment with DNase is effected so as to generate polynucleotide fragments being about 50-100 base pairs in length. Methods of generating polynucleotide fragments having a predetermined approximate length using DNase I digestion are widely available in the literature of the art.

Preferably, the restriction enzyme is Rsa I. The use of Rsa I advantageously enables the generation of blunt-ended polynucleotide fragments which can be cloned into linearized constructs having blunt ends. Linearized constructs having blunt ends can be generated using a suitable blunt-cutting restriction enzyme or can be generated from any non-blunt ended linearized construct by using the appropriate enzymatic reactions to fill in or cleave overhangs.

As is mentioned hereinabove, several approaches can be used to construct the pathway constituent libraries of the present invention.

Libraries of biological pathways can be generated by inducing the expression or over expression of specific biological pathways in appropriate cells and utilizing extracted mRNA (total mRNA or specific subsets) as a template for cDNA synthesis. For example, apoptosis can be induced in cells via treatment with suitable concentrations of apoptosis inducing compounds, such as Taxol and/or doxorubicin, and mRNA species which are triggered as a response to such induction can be collected and utilized as templates for the synthesis of a cDNA library. In a similar manner, cells in which the IGF-I signaling pathway is activated with insulin-like growth factor-I (IGF-I) can also be utilized to prepare a cDNA library.

Libraries of unique cDNA pools can also be generated via subtraction of two mRNA pools each derived from a different cell type or cell state (e.g., normal vs. abnormal).

For example, in cases where constituents of a biological pathways associated with an abnormal phenotype (e.g., cancerous or transformed phenotype) is to be screened against a pathway regulator, a cDNA subtraction library is prepared by subtracting between a cDNA library generated from cells exhibiting a normal phenotype and a cDNA library generated from cells exhibiting the abnormal phenotype.

Pools of polynucleotides derived from such normal and from such abnormal cell types can be reciprocally subtracted from each other to generate libraries enriched for constituents uniquely expressed in one or the other cell type. Further guidance regarding subtraction protocols is available in the literature of the art (see, for example, as disclosed in U.S. Pat. Nos. 5,670,312 and 5,492,807).

An example of a cDNA subtraction library representative of an abnormal phenotype is the small-cell lung cancer cDNA library described in the Examples section below.

The method of the present invention can further be extended to the identification of a specific region of a pathway constituent which binds with the pathway regulator.

In such cases, libraries expressing polynucleotides fragments spanning (overlapped or contiguous) a single constituent of the biological pathway can be utilized for screening. Such a method enables the characterization of a specific region or regions of a target sequence (biological pathway constituent) which binds the regulator.

Although any library type can be utilized successfully with the present invention, as is illustrated by the Examples section which follows, cDNA phage display libraries are particularly advantageously for use with the present invention.

As used herein, the phrase “cDNA phage display libraries” refers to phage display libraries displaying cDNA-encoded molecules. As used herein, the terms “bacteriophage”, and “phage” are interchangeable.

Various types of phages can be used to generate the library. For example, the method may employ lambda phages or M13 phages.

Preferably, the type of phages used to generate the library are M13 phages. Libraries employing M13 phages are widely recognized as being optimal for generating libraries for selection of phage-displayed molecules having a given binding specificity. Ample guidance regarding the construction and/or use of phage display libraries is available in the literature of the art (see for example; Crameri R. and Suter M., 1993, Gene 137:69-75; George R et al., 2000, Drug Discovery Technologies, 4:145-156; Parmley S F. and Smith G P., 1989. Adv Exp Med Biol. 251:215-218; Scott J K. and Smith G P., 1990. Science 249:386-390; Winter J., 1994. Drug and Dev Result. 33:71-89; and U.S. Pat. Nos. 5,223,409; 5,622,699 and 6,068,829).

Such prior art methods of generating cDNA phage display libraries have employed cloning strategies utilizing constructs which do not afford satisfactory control over the reading frame of the chimera comprising N-terminal cDNA encoded peptide and C-terminal phage coat protein relative to upstream leader sequences, as required for suitable expression and display of cDNA-encoded molecules. As a consequence, many phages contained in libraries using prior art constructs will not express cloned peptide-coat protein chimeras, or will express such chimeras translated in an incorrect reading frame. Thus, many phages in such libraries will not display, or will not appropriately display cloned peptide-coat protein chimeras, thereby leading to drastically reduced efficiency in selecting phages displaying molecular targets of the molecular regulator. For example, displayed molecules translated in the wrong reading frame fortuitously having the capacity to bind the molecular regulator will generate false positives, thereby interfering with selection of true cDNA encoded peptides.

In order to circumvent such prior art limitations, the phage display libraries of the present invention are preferably generated using an expression construct system comprising a plurality of expression constructs being for phage display expression of polypeptides, each of the expression constructs having a unique polylinker sequence flanked by a first polynucleotide region encoding a phage leader sequence positioned upstream of the polylinker, and a second polynucleotide region encoding a chimeric polypeptide including a tag sequence fused to a phage coat protein, wherein each unique polylinker is designed to enable cloning of a desired polynucleotide in a unique reading frame combination with respect to the leader sequence and the chimeric polypeptide, such that phage particles expressing the desired polynucleotide cloned in frame to the leader sequence and the chimeric polypeptide can be identified and optionally isolated from a phage particle population transformed with the plurality of expression constructs harboring the desired polynucleotide.

Preferably, the expression construct system employs constructs composed of polylinker sequences inserted into vector pCC11, or a substantially analogous vector, as described in the Examples section which follows.

Preferably, the phage leader sequence used is a gene 3 leader sequence. The gene 3 leader sequence optimally enables the display of chimeras comprising the cloned peptide, the tag, and the phage coat protein by the phage.

Preferably, the phage coat protein is phage coat protein III. Very few copies of phage coat protein III are expressed by the phage, and such proteins are expressed at one of the distal ends of the phage, an elongated structure. As such, phage coat protein III is ideal for presenting cDNA-encoded peptides fused thereto.

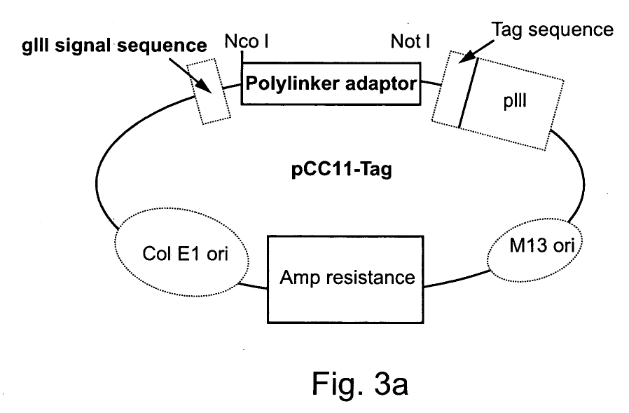

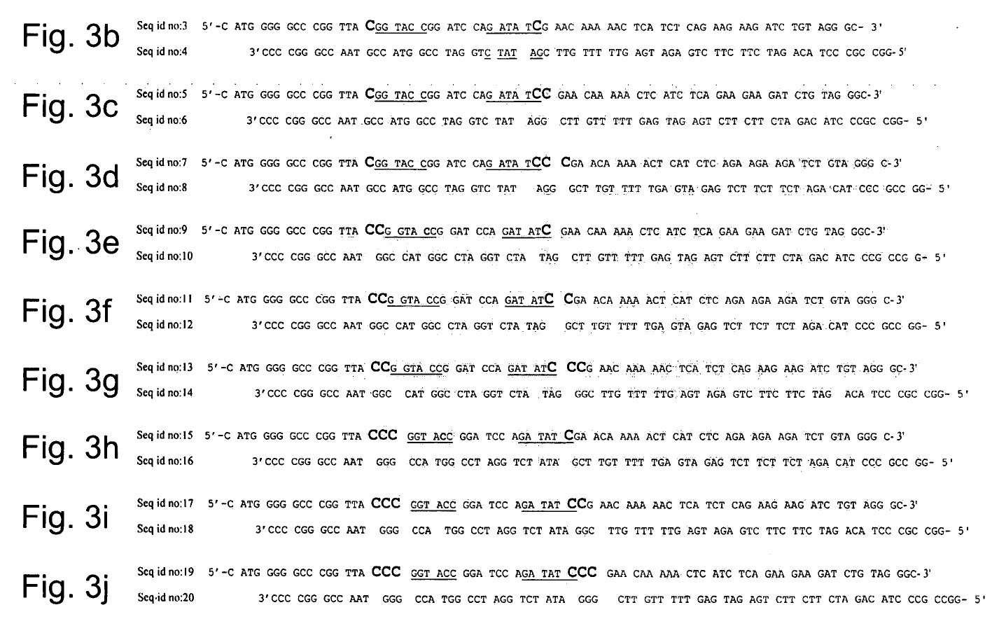

Preferably the polylinker sequences used to generate the library are defined by the sense-antisense nucleic acid sequences defined by SEQ ID NOs: 3-4, 5-6, 7-8, 9-10, 11-12, 13-14, 15-16, 17-18, and 19-20, as shown in FIGS. 3b-j, or polylinker sequences substantially analogous thereto.

As shown in FIGS. 3a-b, and as described in the Examples section which follows, the construct system of the present invention can be used to generate cDNA phage display libraries enabling the in-frame expression of displayed chimeric proteins comprising a cDNA-encoded molecule N-terminally and, C-terminally, a portion of phage coat protein III lacking 195 amino acid residues of its N-terminal. The use of such a phage coat protein III deletion advantageously facilitates display of the fused cDNA-encoded peptide.

The cDNA fragment encoding the cloned peptide is preferably inserted into the EcoR V site of the polylinker sequences. A Iternately, cDNA may be cloned into the BamH I site of the polylinker sequences.

Preferably, the library is generated by genetically transforming bacterial cells with constructs containing cloned peptides, culturing such bacterial cells so as to allow phage production, and harvesting the phage-containing supernatant of such bacterial cultures, for example as described in the Examples section below or as described in the literature of the art, as referenced hereinabove.

Examples of tags fused to the molecules of the library include fluorescent tags, enzyme tags, epitope tags, and affinity tags.

Such tags can be advantageously utilized to isolate and/or visualize selected phages.

Examples of fluorescent tags include green fluorescent protein or blue fluorescent protein.

Examples of enzyme tags include beta-galactosidase, horseradish peroxidase and alkaline phosphatase.

Examples of affinity tags and their corresponding ligands are described further hereinbelow.

Preferably, the affinity tag is a cellulose binding domain. The use of a cellulose binding domain tag facilitates isolation of cellulose binding domain tagged phages from supernatants of phage-infected bacterial cultures.

Recovery of CBD-tagged phage may be effected as follows. Crude phage is obtained from E. coli culture supernatants by centrifugation. The cell free supernatant is passed through a 0.2 μm filter (Sartorius, Germany). A 5 ml aliquot of a 33% slurry of microcrystalline cellulose in sterile double distilled water is added to 100 ml of filtered crude phage, and the mixture is incubated for 30 minutes at room temperature. The cellulose is recovered by brief centrifugation, and the supernatant, containing the unbound phages, is saved. The cellulose pellet is washed with phosphate saline buffer, and the phages are eluted from the cellulose pellet by incubating the pellet with 2 ml of an elution buffer containing 20 mM NaOH and 100 mM NaCl for 10 minutes. Recovered phages are separated from the cellulose by centrifugation and immediately neutralized by addition of 0.2 ml 1M Tris (HCl) pH 7.0. The cDNA sequences of the eluted phages are characterized. This is the primary cDNA library, and it is stored at 4° C. for periods of up to a few days, otherwise aliquots of 0.5 ml are stored in 20% glycerol at −80° C. for longer periods.

Once generated, the library is preferably screened for a molecule capable of specifically binding the molecular regulator of the biological pathway so as to thereby characterize the molecular target of the molecular regulator, as described above.

Screening the library is preferably effected by attaching the molecular regulator to a substrate, exposing the molecular regulator to the library, to thereby obtain complexes each composed of the molecular regulator and a molecule displayed by the library, and isolating the complexes, as described in the following Examples section. Once the complexes are isolated the method preferably further comprises identifying the molecule associated with the molecular regulator in the complexes.

Attachment of molecular regulators to substrates can be effected using a variety of methods.

For example, molecular regulators may be attached to a substrate to which a molecule capable of specifically binding the molecular regulator is attached. Alternately, molecular regulators are produced as chimeras comprising an affinity tag and are attached to a substrate to which a specific ligand of the affinity tag has been conjugated, as described in detail in the Examples section below. Alternately, molecular regulators are passively coated onto a suitably adherent substrate, such as a suitably adherent plastic surface.

As used herein, the phrase “affinity tag” refers to a molecule which can be conjugated to the molecular regulator and for which a specific ligand is available.

Examples of affinity tags include, but are not limited to, a histidine tag [(His)₆], a cellulose binding domain, a biotin molecule, a streptavidin molecule, an epitope tag, a DNA-binding domain, and blue fluorescent protein. Specific ligands of such tags include anti histidine tag antibody, cellulose, streptavidin, biotin, an epitope tag specific antibody, a DNA-binding domain specific transcription factor domain, and an anti blue fluorescent protein antibody, respectively. Types of affinity tags, their specific ligands and methods of using such are described in extensive detail in the literature of the art.

Preferably, the affinity tag and ligand thereof employed are biotin and streptavidin, respectively.

Types of substrates suitable for attachment of the molecular regulator include magnetic beads, or suitable plastic surfaces such as ELISA plates such as MaxiSorp Nunc MicroWell ELISA plates. As described in the Examples section below, the molecular regulator can be passively attached to such ELISA plates for screening the library or can be attached to streptavidin-conjugated magnetic beads.

Exposure of the substrate-attached molecular regulator to the library and isolation of complexes may be suitably effected as described in the Examples section, below, or using analogous techniques described in the literature of the art.

Isolation of complexes is preferably effected by thoroughly washing off unbound phages from the substrate. Such isolation preferably further comprises using the isolated substrate-bound phages to infect bacteria so as to propagate such phages, and cloning individual phages.

Once individual phages are cloned, the cDNA cloned in such phages is preferably PCR amplified, thereby enabling nucleic acid sequencing, or further manipulation thereof. Alternately, the cloned cDNA can be PCR amplified during any of the prior steps after sufficiently washing off free phages from the substrate.

Preferably, individual cloned phages are tested for their capacity to specifically bind the molecular regulator. This can be effected by spotting phages on PVDF or nitrocellulose membranes, reacting the membranes with tagged molecular regulator and detecting specific retention of the tag on the membrane. Preferably the tag used for such application is a histidine tag.

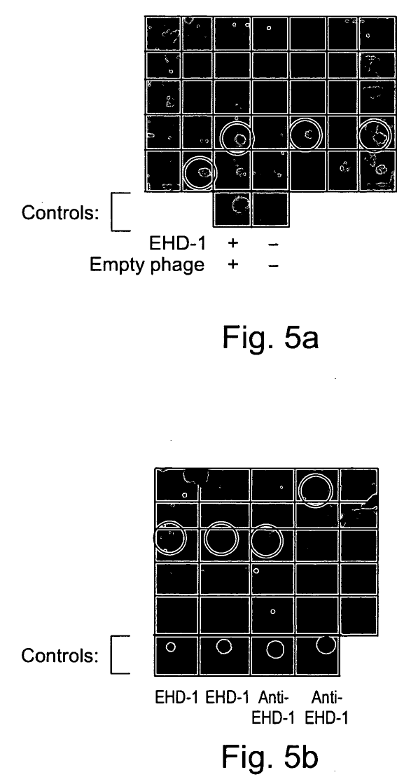

Preferably, the tag is detected via an enzyme linked assay using an enzyme-conjugated tag specific ligand, such as horseradish peroxidase conjugated anti tag antibody, as is illustrated in FIGS. 5a-b and described in the Examples section.

Hence, molecular targets encoded by cDNA sequences individually cloned phages specifically binding the molecular regulator of the biological pathway have an optimal probability of being capable of regulating the biological pathway.

Thus, the above described aspect of the present invention enables isolation and characterization of molecular targets of molecular regulators. It will be appreciated that once a molecular target is isolated, it can be used along with the molecular regulator to identify substances having the same binding specificities and/or regulatory capacities as the molecular target, but having physical, chemical and/or biological characteristics, required, for example, for optimal pharmacological activity, or for optimal drug development not exhibited by the molecular regulator.

Thus, according to another aspect of the present invention there is provided a method of uncovering a putative functional analog of a molecular regulator of a biological pathway.

As used herein, the phrase “functional analog of a molecular regulator” refers to a substance having essentially similar molecular target binding capacity as the molecular regulator and/or essentially similar capacity to regulate a biological pathway as the molecular regulator.

The method is effected by incubating the molecular regulator and its respective molecular target in the presence of each of a plurality of distinct substances to thereby identify a substance of the plurality of distinct substances which is capable of competing with the molecular regulator for binding with the molecular target.

As is described in the Examples section which follows, screening for functional analogues can be effected using various types of competition assays.

According to one preferred embodiment, the method is effected using a competition assay in which the molecular regulator comprises a detectable tag, and identifying the substance capable of competing with the molecular regulator for binding of the molecular target is effected by detecting dissociation of the detectable tag from the molecular target. With respect to this embodiment, the method is preferably effected by attaching the complex to a substrate via the molecular target, exposing the complex to each of the plurality of substances, and washing off non substrate-bound molecules from the substrate prior to detecting dissociation of the detectable tag from the molecular target of the complex.

According to another preferred embodiment, the method is effected using a competition assay in which the molecular regulator comprises a detectable tag, and identifying the substance capable of competing with the molecular regulator for binding of the molecular target is effected by detecting dissociation of the detectable tag from the molecular regulator of the complex. With respect to this embodiment, the method is preferably effected by attaching the complex to a substrate via the molecular regulator, exposing the complex to each of the plurality of substances, and washing off non substrate-bound molecules from the substrate prior to detecting dissociation of the detectable tag from the molecular regulator of the complex.

According to a further embodiment, each of the plurality of distinct substances comprises a detectable tag, and identifying the substance capable of competing with the molecular regulator for binding of the molecular target is effected by detecting association of the detectable tag with the molecular target. With respect to this embodiment, the method is preferably effected by attaching the complex to a substrate via the molecular target, exposing the complex to each of the plurality of substances, and washing off non substrate-bound molecules from the substrate prior to detecting association of the detectable tag with the molecular target.

According to yet a further embodiment, each of the plurality of distinct substances comprises a fluorophore tag capable of being a member of a fluorescence resonance energy transfer (FRET) pair and the molecular regulator comprises the complementary fluorophore of the FRET pair, and identifying the substance capable of competing with the molecular regulator for binding of the molecular target is effected by detecting FRET between such fluorophores, the amount of FRET being indicative of the capacity of the tested substance to specifically bind the molecular regulator.

According to an additional embodiment, the molecular target comprises a fluorophore tag capable of being a member of a fluorescence resonance energy transfer (FRET) pair and the molecular regulator comprises the complementary fluorophore of the FRET pair, and identifying the substance capable of competing with the molecular regulator for binding of the molecular target is effected by detecting loss of FRET between such fluorophores.

With respect to FRET-based embodiments, the method is preferably effected by attaching the complex to a substrate via the molecular target, exposing the complex to each of the plurality of substances, and monitoring changes in FRET specific fluorescence.

Examples of FRET pairs include fluorescein and tetramethylrhodamine, IAEDANS and fluorescein, EDANS and DABCYL, fluorescein and fluorescein, BODIPY FL and BODIPY FL, fluorescein and QSYTM-7, dansyl ([dimethylamino]naphthalene-1-sulfonyl) and tryptophan.

Ample guidance regarding fluorophore selection, methods of linking fluorophores to various types of molecules such as polypeptides or polynucleotides, is available in the literature of the art (see, for example: Gakamsky D. et al., “Evaluating Receptor Stoichiometry by Fluorescence Resonance Energy Transfer,” in “Receptors: A Practical Approach,” 2nd ed., Stanford C. and Horton R., eds., Oxford University Press, UK. (2001); Richard P. Haugland, Molecular Probes: Handbook of Fluorescent Probes and Research Chemicals 1992-1994 (5th ed., 1994, Molecular Probes, Inc.); U.S. Pat. No. 6,037,137 to Oncoimmunin Inc.; Hermanson, (1995) Bioconjugate Techniques, Academic Press New York, N.Y.; Kay M. et al., 1995. Biochemistry 34: 293; Stubbs, et al. (1996) Biochemistry 35: 937; U.S. Pat. No. 6,350,466 to Targesome, Inc.).

Guidance for using FRET in high-throughput screening assays can be obtained in the literature of the art (see for example Stenroos K. and Hurskainen P. 1998. Cytokine 495:5; Kane S A. et al., 2000. Anal. Biochem. 278:29).

Various types of detectable tags can be used according to this aspect of the present invention.

Examples of suitable detectable tags include fluorescent tags, enzyme tags, epitope tags, and affinity tags.

Examples of suitable fluorescent tags include green fluorescent protein, blue fluorescent protein, FITC and rhodamine.

Examples of suitable enzyme tags include beta-galactosidase, horseradish peroxidase and alkaline phosphatase.

Examples of suitable epitope tags and affinity tags include such tags which can be detected by an enzyme-conjugated or fluorescent tag conjugated anti tag antibody. Preferably, the enzyme-conjugated to the antibody is horseradish peroxidase. Detection of molecules via horseradish peroxidase can be suitably effected, for example, as described in the Examples section below.

Methods of tagging molecules and detecting such molecules in a broad variety of contexts are extensively detailed in the literature of the art.

According to yet a further embodiment, identifying a substance of the plurality of distinct substances capable of competing with the molecular regulator for binding of the molecular target can be efficiently effected using surface plasmon resonance methods, such as, for example, BiaCore apparatus-based methods. Methods of using surface plasmon resonance to detect association or dissociation of molecules are highly standardized and may easily be applied to this aspect of the present invention using the ample guidance provided by the literature of the art.

Since this aspect of the present invention may be effected using fluorescence detection or surface plasmon resonance detection of intermolecular association/dissociation, the method is advantageously effected using high throughput methods, for example, as described in the Examples section below or as extensively detailed in the literature of the art (see, for example, Kyranos J N. et al., 2001. Curr Opin Drug Discov Devel. 4(6):719); Hunter D., 2001. J Cell Biochem Suppl. Suppl 37:22; Kerns E H., 2001. J Pharm Sci. 90(11):1838).

The method according to this aspect of the present invention can be used to uncover putative functional analogs of any type of molecular regulator.

Preferably the method is used to uncover putative functional analogs of polypeptide regulators. Since the great majority of constituents of signaling pathways are polypeptides, and thus are involved in the overwhelming majority of intermolecular interactions occurring between constituents of biological pathways, the capacity of this aspect of the present invention to uncover functional analogs of polypeptides can be very advantageously applied to generate putative functional analogs of the extremely broad and potent range of available, and theoretically available, polypeptide regulators. Examples of polypeptide constituents of biological pathways include, for example, cell surface receptors, second messengers such as, for example, kinases, and phosphatases, as well as transcription factors.

Thus, functional analogs of polypeptide regulators can be used to treat a very wide variety of disorders characterized by biological pathways, as described hereinabove.

Since the method can be used to uncover putative functional analogs of polynucleotide molecular regulators, the method can be used to uncover putative functional analogs of a gene regulatory element such as a promoter. Functional analogs of promoters can be advantageously employed, for example, as blocking reagents functioning to prevent binding of transcription factors to promoters. Such a capacity can be usefully applied to treating diseases associated with gene overexpression, as described hereinabove, in the following Examples section, and in the extensive literature of the art.

Preferably, promoters for which the method can be used to generate putative functional analogs are vascular endothelial growth factor (VEGF) promoters or apoptotic protease activating factor-1 (APAF-1) promoters.

Functional analogs of VEGF promoters can be advantageously used to block diseases associated with excess IGF-I receptor activated signaling, such as various cancers.

Molecular regulators used according to this aspect of the present invention are preferably generated as described in PCT No. WO0138569A1 or as described in the following examples section.

Preferably, the primers used to generate VEGF promoter via PCR amplification correspond to SEQ ID NOs: 1 and 2.

This aspect of the present invention can be most advantageously be used to uncover putative functional analogs of molecular regulators having extremely useful characteristics not possessed by such molecular regulators.

For example, the method can be used to uncover putative molecular regulators having a lower molecular weight and/or a smaller volume than that of the molecular regulator. Furthermore, the method can be used to uncover putative non-polypeptide functional analogs of polypeptide regulators. Such capacities can be very advantageously employed to uncover putative functional analogs having properties overcoming various major drawbacks of molecular regulators. For example, molecular regulators, while being potentially useful as pharmacological agents are often too large to display optimal biodistribution and/or pharmacokinetic parameters. Such drawbacks can be very effectively overcome by functional analogs of such molecular regulators having smaller, optimal dimensions relative to those of such molecular regulators. Furthermore, polypeptide regulators potentially useful as pharmacological agents display significant drawbacks due to their polypeptidic composition. For example, polypeptide regulators display unsatisfactory in-vivo stability following therapeutic administration due to physiological mechanisms acting to degrade polypeptides, or display unsatisfactory stability during storage due to the susceptibility to rapid oxidation damage of polypeptides. Such drawbacks can be potently overcome by non-polypeptide putative functional analogs of such molecular regulators having desired physico-chemical properties generated according to this aspect of the present invention.

Thus, by using molecules shown to be capable of regulating a biological pathway to identify putative functional analogs of such molecular regulators, this aspect of the present invention is superior to all prior art methods which uncover putative functional analogs of molecules which have only been shown to bind constituents of a biological pathway.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-111 Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes 1-111 Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); Biotechnol Bioeng Oct. 5, 1999; 65(1):1-9 Prediction of antisense oligonucleotide binding affinity to a structured RNA target. Walton S P, Stephanopoulos G N, Yarmush M L, Roth C M; Prediction of antisense oligonucleotide efficacy by in vitro methods. O Matveeva, B Felden, A Tsodikov, J Johnston, B P Monia, J F Atkins, R F Gesteland & S M Freier Nature Biotechnology 16, 1374-1375 (1998); all of which are incorpotaed by reference as if fully set forth herein. Other general references are provided throughout this document. The procedures therein are believed to be well known in the art and are provided for the convenience of the reader. All the information contained therein is incorporated herein by reference.

### Example 1

### Identification and Isolation of Lead Peptide Regulators of IGF-I Receptor Signaling

Many disease states, such as cancer, are associated with IGF-I receptor signaling. Thus, identification and isolation of signaling intermediates of IGF-I receptor signaling pathways is essential for strategies aiming to identify compounds capable of regulating IGF-I receptor signaling, and hence being useful to treat diseases such as cancer. To date, however, there are no satisfactory methods of identifying and isolating signaling intermediates of IGF-I receptor induced transduction pathways.

In order to fulfill this important need, the present inventors have devised methods of identifying and isolating signaling intermediates of IGF-I receptor induced signal transduction pathways, as follows.

Materials and Methods:

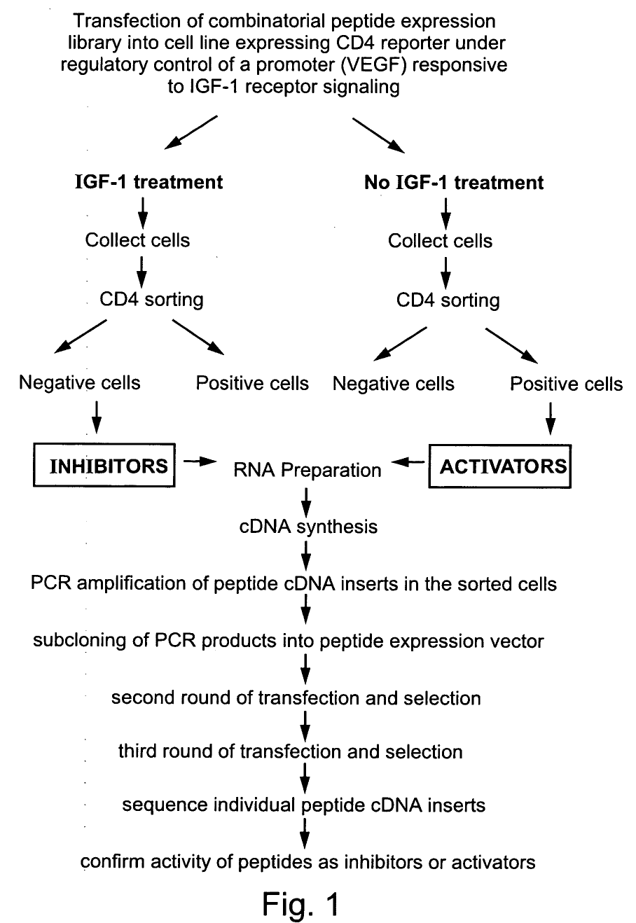

Lead peptide regulators of the IGF-I receptor signaling pathway are isolated essentially as described in patent application PCT/IL00/00680. Lead peptide regulators capable of regulating IGF-I receptor induced signaling are identified by expressing a peptide library and a VEGF promoter-driven reporter gene in NIH-3T3 cells expressing human IGF-I receptor. IGF-I receptor induced signaling is monitored in these cells via expression of the reporter gene. A screening system used to isolate peptide regulators of the IGF-I receptor signaling pathway is outlined in FIG. 1.

cDNA phage display libraries displaying IGF-I receptor signaling intermediates, or portions thereof, are generated from cells expressing such intermediates, or portions thereof, and such display libraries are screened to identify IGF-I receptor signaling intermediates, or portions thereof, specifically bound by selected peptide regulators of IGF-I receptor signaling.

Lead peptide regulators are tagged with a detection marker and biotin, and are attached to streptavidin-conjugated solid substrates. Solid substrates used are ELISA plates or magnetic beads. Substrate-bound lead peptide regulators are reacted with the cDNA phage display libraries to form specifically bound phage-lead peptide regulator complexes. Complex-bound substrates are extensively washed, and remaining phages complexed with lead peptide regulators are used to infect TG1 bacteria to recover, clone and propagate such phages. This selection process is repeated three times for enrichment of phages specifically bound to lead peptide regulators.

Preparation of IGF-I Receptor Signaling Reporter Cells:

NIH 3T3 cells expressing human IGF-I receptor or the human breast cancer cell line T47D are transfected with a DNA expression vector encoding a GFP or CD4 reporter gene under the control of the human vascular endothelial growth factor (VEGF) promoter, and an antibiotic resistance gene. Introduction of reporter gene expression vector and the random peptide expression library into reporter cells is performed according to the calcium phosphate transfection method. Aliquots of 2.5-7×10⁵ cells are plated in 10 cm dishes 16-24 hours prior to transfection. Fresh medium is added to the cells immediately prior to transfection, and a transfection mixture including 0.5 ml of 20 mM HEPES pH 7.05, 120 mM CaCl₂, 2-10 μg transforming DNA, 0.5 ml of 50 mM NaCl, 2 mM KCl, 0.3 mM Na₂HPO₄, 1.25 mM sucrose and 5 mM HEPES pH 7.05 is added to the cells, and the medium is replaced following overnight incubation. Forty-eight hours following transfection cells are harvested for further manipulation.

Construction of Expression Libraries of Peptides Tested for Regulator Activity:

Human placental mRNA is used to prepare a cDNA clone pool. A sample containing 5 μg of total placental RNA, 2 μl of oligodT (10 mM) in final volume of 10 μl is incubated at 80° C. for 10 minutes and immediately chilled on ice. Four microliters of 5× reaction buffer (250 mM Tris-HCl, pH 8.3, 375 mM KCl, 15 mM MgCl₂), 2 μl of 100 mM DTT, and 1 μl dNTPs (10 mM dTTP, dATP, dCTP and dGTP) are added to the sample to a final volume of 19 μl. The sample is incubated at 42° C. for 10 minutes. Afterwards, 200 units reverse transcriptase (Superscript II, GibcoBRL) is added to the sample and the sample is further incubated at 42° C. for 2 hours.

cDNAs encoding human IGF-I receptor, human IRS-I and human EHD-1 are isolated by PCR reaction using specific primers. PCR reaction mixtures included 5 μl of 10× buffer (200 mM Tris HCl, pH 8.4, 500 mM KCl), 2 μl of 10 mM dNTP mixture (10 mM of each), 1 mM MgSO₄, 0.5 μM of each primer, 5 μl DMSO and 1 μl Taq DNA polymerase (Platinum Pfx DNA polymerase from GibcoBRL) in a final volume of 50 μl. The thermocycling reaction included a denaturation step of 95° C. for 5 minutes followed by 30 cycles, each including: a denaturation step −95° C., 1 minute, a hybridization step −68° C., 10 seconds, 67° C., 10 seconds, 66° C., 10 seconds, 65° C., 10 seconds, 64° C., 10 seconds, 10 seconds, 62° C., 10 seconds and 60° C., 10 seconds; and an elongation step of 68° C., 5 minutes; followed by a final elongation step of 72° C., 10 minutes. Resultant PCR products are analyzed and purified on a TAE agarose gel.

These PCR products are each digested with DNase to generate 50-100 base pair DNA fragments. The 5′ overhangs of the fragments are blunt-ended using Klenow polymerase. The resultant DNA fragments are each ligated into the shuttle vectors [pQBI-50 pfA for N-terminal cloning, and pfC (Quantum Biology Inc., USA) for C-terminal cloning in all three open reading frames] to generate a signaling intermediate peptide expression library. The ligation reaction is performed in a final volume of 20 μl and contains 20 nanograms of vector DNA digested with EcoRV, 10 nanograms of DNA fragments in 50 mM Tris HCl, 10 mM MgCl₂, 10 mM DTT, 1 mM ATP, 5% polyethylene glycol 4000 and 25 μg/ml BSA. Each reaction is incubated for 5 hours at 20° C. Each library generated contained approximately 3,000 different DNA fragments. Each library is separately introduced into the IGF-I receptor reporter cell line described hereinabove via the calcium phosphate transformation technique, as also described hereinabove. The final library expression constructs included the various DNA products in fusion to coding sequence both under the transcriptional control of a CMV or T7 promoter.

Forty eight hours following transfection with the peptide expression library, cells are either treated or not treated with the inducer (IGF-I). IGF-I-treated cells not expressing the reporter gene contain an inhibitory peptide, and non-IGF-I-treated cells expressing the reporter gene contain an inhibitory peptide.

Cells using a GFP reporter are sorted and isolated via flow cytometry, and cells using a CD4 reporter are sorted and isolated with magnetic beads conjugated with anti CD4 antibodies. Total RNA is extracted from these cells using a commercially available RNA preparation kit (EZ-RNA of Biological Industries, Israel). An RT-PCR reaction is performed, using oligonucleotide primers flanking the multiple cloning site of the library vector to isolate DNA inserts encoding lead regulator peptides.

Results:

Molecular targets of compounds capable of regulating IGF-I receptor induced transduction pathways are identified and isolated.

Conclusion:

The present method can be used to identify molecular targets expressed in cancer cells targeted by peptides capable of regulating IGF-I receptor transduction pathways. The identification and isolation of such molecules can be used to design improved compounds capable of treating diseases such as cancer.

### Example 2

### Identification and Isolation of Lead Peptide Regulators of Apoptosis

The capacity to induce apoptosis of diseased cells, such as cancer cells, represents an attractive strategy for treatment of diseases such as cancer. One promising approach to induce apoptosis in diseased cells would be to identify and isolate molecules involved in regulation of apoptosis, and using such molecules to select peptides capable of regulating apoptosis in cells, such as cancer cells. However, to date, no satisfactory methods exist for identification and isolation of molecules involved in regulation of apoptosis.

In order to fulfill this important need, the present inventors have devised methods of identifying and isolating molecules involved in regulation of apoptosis in cells such as cancer cells, as follows.

Materials and Methods:

General Protocol:

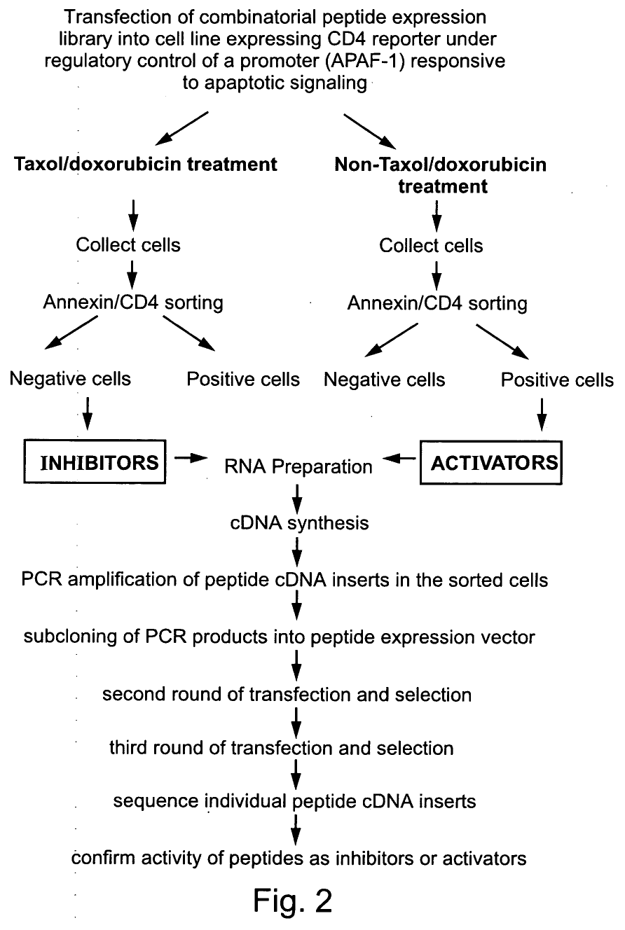

Lung cancer cells or reporter lung cancer cells expressing a reporter gene under the regulatory control of a p53-activated promoter are transfected with peptide expression libraries for identification of lead regulator peptides for regulation of apoptosis. Cells expressing peptides are treated or not treated with a pro-apoptotic treatment and apoptosis is assessed in these cells by monitoring reporter gene expression or via an annexin V binding assay. The screening system used for selection of lead peptide regulators of apoptosis is outlined in FIG. 2.

Reporter Cells:

H1299, H522 or H23 cells are stably transfected to express a human CD4 reporter gene under the transcriptional regulation of the apoptotic protease activating factor-1 (APAF-1) promoter, a direct transcriptional target of p53 which activates apoptosis-inducing caspases (Braton and Cohen, 2001. Trends Pharmacol Sci. 22:306-315; Moroni et al., 2001. Nat Cell Biol. 3:552-558; Shinoura et al., 2001. Int J Cancer 93:252-261; Cecconi et al., 1998. Cell 18:94-104; Soengas et al., 2001. Nature 409:141-144).

Peptide Expression Libraries:

Total cDNA is prepared from human non-small cell lung tumor specimens and from normal lung tissue. Tumor-subtracted and normal tissue-subtracted cDNA pools are generated using Select cDNA Subtraction Kit (Clontech), according to the manufacturer's instructions. Each cDNA pool is PCR amplified in a reaction mix including 5 μl of 10× buffer (200 mM Tris HCl, pH 8.4, 500 mM KCl), 2 μl of 10 mM dNTP mixture (10 mM of each), 1 mM MgSO₄, 0.5 micromolar of each primer, 5 μl DMSO and 1 μl Taq DNA polymerase (Platinum Pfx DNA polymerase from GibcoBRL) in a final volume of 50 μl. The thermocycling reaction includes a denaturation step of 95° C. for 5 minute; followed by 30 cycles, each including: a denaturation step −95° C., 1 minute, a hybridization step −68° C., 10 seconds, 67° C., 10 seconds, 66° C., 10 seconds, 65° C., 10 seconds, 64° C., 10 seconds, 63° C., 10 seconds, 62° C., 10 seconds and 60° C., 10 seconds; and an elongation step of 68° C., 5 minutes; followed by a final elongation step at 72° C., 10 minutes. Resultant PCR products of both subtracted libraries are separately digested with DNAse I to generate 50-100-base pair DNA fragments. Klenow polymerase is used to fill in 5′ overhangs generated following DNase I digestion. The resultant DNA fragments are each ligated into shuttle vectors [pQBI-50 pfA for N terminal cloning and pfC for C terminal cloning (Quantum Biology Inc. USA) in all three open reading frames] to generate an expressed peptide library. The ligation reaction is performed in a final volume of 20 μl and contains 20 nanograms of the vector DNA digested with EcoRV, 10 nanograms of the DNA fragments in 50 mM Tris HCl, 10 mM MgCl₂, 10 mM DTT, 1 mM ATP, 5% polyethylene glycol 4000 and 25 μg/ml BSA. Each reaction is incubated for 5 hours at 20 ° C. Each library generated contains approximately 10,000 different DNA fragments.

The peptide expression libraries are then transfected into reporter cells and the effects on apoptosis in these cells is monitored.

Transfection:

Transfection of the reporter DNA vectors and the peptide library into cells is performed via the calcium phosphate transfection method. Aliquots of 2.5-7×10⁵ cells are plated in 10 cm diameter culture dishes 16-24 hours prior to transfection. Immediately prior to transfection, fresh medium is added to the cells. A DNA transfection mixture including 0.5 ml of 20 mM HEPES pH 7.05, 120 mM CaCl₂, 2-10 μg DNA of interest, 0.5 ml of 50 mM NaCl, 2 mM KCl, 0.3 mM Na₂HPO₄, 1.25 mM sucrose and 5 mM HEPES pH 7.05 is added to the cells, and medium is replaced the following morning.

Identification and Isolation of Apoptosis-regulatory Peptides:

Forty eight hours following cell transfection with peptide expression libraries, cells are treated or not treated with Taxol or doxorubicin, pro-apoptotic stimuli. Non-treatment is also effected using an apoptosis-sensitizing concentration of Taxol or doxorubicin (50-100 nM range). After 18 hours, cells are sorted according to annexin staining or CD4 reporter expression using magnetic beads conjugated to annexin V, or using biotinylated anti-CD4 antibodies and streptavidin-conjugated magnetic beads. Alternately, annexin V conjugated to FITC (BioVision, USA) is used to sort cells via FACS and to monitor cell apoptosis by fluorescent microscopy.

Taxol/doxorubicin-treated cells which are annexin V-binding or CD4 reporter negative are selected as containing lead peptide inhibitors of apoptosis.

Non-Taxol/doxorubicin-treated cells or cells treated with sensitizing doses thereof only which are annexin V positive or CD4 reporter positive are selected as containing lead peptide activators of apoptosis.

Recovery of lead peptide regulators of apoptosis is effected by extracting total RNA from selected cells using EZ-RNA RNA preparation kit (Biological Industries, Israel). DNA inserts encoding lead peptide regulators are cloned by RT-PCR using oligonucleotide primers flanking the multiple cloning site of the library vector.

Multiple rounds of transfection and selection are performed to enrich apoptosis regulatory peptides, and individual selected lead peptide regulators of apoptosis are tested to analyze their ability to regulate apoptosis.

Results:

Peptides capable of regulating apoptosis in cancer cells are identified and isolated.

Conclusion:

The present methods can be used to identify peptides capable of regulating apoptosis in cancer cells. The identification and isolation of such peptides can be used to design improved compounds capable of treating diseases such as cancer.

### Example 3

### Identification an Disolation of Peptide Regulators of Bacterial Growth and Survival

Increasing bacterial resistance to antibiotics is responsible for increasingly widespread mortality and morbidity and thus has created a critical need for new antibiotics. However, to date, no satisfactory methods of identifying peptides having antibacterial activity exist. In order to fulfill this vital need, the present inventors have devised a method of identifying and isolating antibacterial peptides, as follows.

Materials and Methods:

A peptide cDNA expression library is generated from the whole bacterial genome as a source of potential inhibitors to bacterial growth and is used to select for lead peptides with bactericidal activity. The peptide library is expressed under the control of an inducible promoter, such as a Lac Z promoter or araBAD (Invitrogen) that contains a promoter that allows tight regulation of gene expression using different carbon sources or selection conditions. The peptide library is introduced into the bacteria of interest, and selection of active lead peptides is done in normal or inducible media. Bacteria growing normally in complete media and whose growth is inhibited in inducible media are selected as expressing a library peptide with antibacterial activity. The peptide-encoding sequences are retrieved by PCR amplification with specific primers complementary to insert-flanking sequences, similarly to the relevant stages of the scheme outlined in FIGS. 1 and 2. More specifically, such peptides can be identified, as follows.

cDNA of Staphylococcus aureus is generated using standard protocols. The DNA is digested with DNase to generate 50-100-base pair fragments and Klenow polymerase extension reaction is used to fill-in 5′ overhangs. The resultant DNA fragments are ligated into shuttle vectors based on pBAD/Myc-His of Invitrogen. The ligation reaction is performed in a final volume of 20 μl containing 20 nanograms of vector DNA digested with EcoRV, 10 nanograms of the DNA fragments in 50 mM Tris HCl, 10 mM MgCl₂, 10 mM DTT, 1 mM ATP, 5% polyethylene glycol 4000, and 25 μg/ml BSA. The reaction is incubated for 5 hours at 20° C. The library generated contains over 500,000 different DNA fragments. The library is introduced back into Staphylococcus by electroporation. Different dilutions of transformed bacteria are grown in medium in the absence or presence of arabinose to block or induce transcription, respectively. Peptide-encoding cDNA sequences are recovered from bacteria displaying differential growth in these media for further analysis. The selected DNA constructs are further analyzed and fusion protein blue fluorescent protein-lead peptide regulator chimeras are purified for drug development and for screening protein complexes.

Results:

Peptides capable of increasing or inhibiting bacterial growth or capable of killing bacteria are identified and isolated.

Conclusion:

The method described herein can therefore be used to identify and isolate peptides useful as antibiotics or to accelerate growth of bacteria in culture, and hence to enhance recombinant protein expression yields by cultured bacteria.

### Example 4

### Identification and Isolation of Cellular Protein Targets of a Polypeptide Regulator (EHD-1) of a Signaling Pathway (IGF-I Receptor-activated)

Transduction pathways mediated by IGF-I receptor are involved in a broad range of cellular and physiological processes associated with human diseases such as cancer. EHD-1 is a polypeptide regulator mediating endocytosis induced by binding of ligands to IGF-I receptor. Protein targets of EHD-1 represent attractive targets for identification and isolation of compounds capable of regulating transduction pathways mediated by IGF-I receptor, and hence of compounds useful for treatment of disease states associated with transduction pathways induced via IGF-I receptor, such as cancer. However, to date, no satisfactory methods exist for identification and isolation of cellular protein targets of polypeptide regulators, such as EHD-1, exist. In order to fulfill this important need, the present inventors have devised a method of identifying and isolating lead protein targets of polypeptide regulators, as follows.

Materials and Methods:

Generation of cDNA Phage Display Library Expressing Cellular Protein:

Full or partial length cDNA phage display libraries with a titer of over 10¹² phages per ml were prepared from human breast cancer cells (T47D and MCF7 cell lines) as follows. Total RNA was prepared from cells using the EZ-RNA kit (Clontech) according to the manufacturer's instructions. A sample containing 10 μg total RNA, 2 μl of 10 μM cDNA synthesis primer in a final volume of 10 μl was incubated at 80° C. for 10 minutes and immediately chilled on ice. Four microliters of 5× buffer (250 mM Tris-HCl pH 8.3, 375 mM KCl, 15 mM MgCl₂), 2 μl of 100 mM DTT, and 1 μl of 10 mM dNTPs (dTTP, dATP, dCTP and dGTP) were added to the sample to a final volume of 19 μl. The sample was incubated at 42° C. for 10 minutes, after which 200 units reverse transcriptase (Superscript II; GibcoBRL) were added to the sample. The sample was further incubated at 42° C. for 2 hours.

Phage Display Libraries for Expression of Cellular Proteins:

cDNA was digested with RsaI to generate blunt-ended DNA fragments which were cloned into E coRV-digested pCC11 phagemid vectors (FIG. 3a) using a set of nine polylinker adaptors (FIGS. 3b-j; SEQ ID NOs: 3-4, SEQ ID NOs: 5-6, SEQ ID NOs: 7-8, SEQ ID NOs: 9-10, SEQ ID NOs: 11-12, SEQ ID NOs: 13-14, SEQ ID NOs: 15-16, SEQ ID NOs: 17-18, and SEQ ID NOs: 19-20, respectively), allowing in-frame expression of a chimeric polypeptide comprising the cDNA encoded sequences fused to a detectable tag and a C-terminal segment of phage coat protein III. Ligation was performed using T4 DNA ligase (New England BioLabs) in a reaction mix containing 50 mM Tris HCL pH 8.0, 1 mM DTT, 10 mM A TP a nd 1 mM MgCl₂. The reaction was incubated overnight at 16° C. Ligation products were transformed into XL-1 blue bacteria (Stratagene, La Jolla, Calif.) by electroporation. Transformants were plated onto 2× YT agar plates containing 100 μg/ml ampicillin and 1% glucose, and were grown overnight at 25° C. for library amplification. The colonies were scraped off the plates into 2× YT medium and the amplified library stock was stored at −80° C. following addition of sterile glycerol to 15%.

Production of Polypeptide Regulator (EHD-1):

Recombinant EHD-1 protein fused to a histidine tag for protein purification and an N-terminal epitope tag for protein detection was expressed in bacteria transformed with the T7 promoter driven expression vector pRSET (Invitrogen) comprising an insert encoding EHD-1 (FIG. 4a).

Identification and Isolation of Target Ligands of Polypeptide Regulator (EHD-1):

EHD-1 was substrate-immobilized by adding 1 μg/ml histidine-tagged EHD-1 in PBS to ELISA plates (MaxiSorp Nunc MicroWell) followed by incubation at room temperature for 2 hours. The solution was aspirated and the coated surface was blocked by the addition of 200 μl of 1% (w/v) BSA solution in NaHCO₃ pH 8.5, 1% gelatin in phosphate buffer saline solution, or 2% BSA in phosphate buffer saline solution, and incubating the blocking mixture for 1 hour at room temperature. Plates were washed three times with PBS containing 0.05% Tween.

Aliquots of phage library (10¹⁰ phages) were added to the wells, and the mixture was incubated for one hour at room temperature to enable binding of specific phages to immobilized polypeptide regulator (EHD-1).

The plates were washed extensively, a 100 μl aliquot of 50 mM glycine pH 2.0 was added to each well, and the plates were incubated for 15 minutes at room temperature. The glycine solution was transferred to new tube and the solution was neutralized by raising the pH to pH 8.0 by addition of 25 ml 1M Tris-HCL pH 8.0. A 1 ml inoculum of mid-log phase TG1 bacteria culture was added to the plates, and the infection mixture was incubated at 37° C. for 30 minutes. Following incubation, infected cells were plated on LB agar plates supplemented with ampicillin. The protocol used for selection of phages specifically interacting with polypeptide regulator (EHD-1) is schematically demonstrated in FIG. 4b.

Three such selections were performed to enrich the population of phages that bind the polypeptide regulator (EHD-1).

Samples of 500 individual selected phages were spotted onto PVDF or nitrocellulose membranes and the membranes were reacted with histidine-tagged EHD-1. Detection of phages specifically binding EHD-1 was performed by Western immunoblotting assay using anti His antibodies conjugated to horseradish peroxidase (HRP) and a developing assay using a fluorescent HRP substrate. Displayed cellular proteins potentially capable of regulating EHD-1 function were identified by sequencing phage displayed DNA.

This selection process was performed three times to enrich the phage population binding specifically to the polypeptide regulator (EHD-1).

Experimental Results:

Phages displaying protein capable of specifically binding the regulator molecule (EHD-1) were identified, as shown in FIG. 5a-b.

Conclusion:

The aforementioned method can be used to identify and isolate protein targets of polypeptide regulators, such as EHD-1. Such lead protein targets are useful for identification of compounds capable of regulating signaling pathways, such as signaling pathways activated by IGF-I receptor, and hence for identification of compounds useful for treatment of disease states, such as disorders associated with IGF-I receptor signaling, such as cancer.

### Example 5

### Identification and Isolation of Polypeptides Interacting with a p53 Inducible Promoter

Binding of proteins to DNA or RNA molecules regulates various processes, including DNA replication, RNA transcription, protein translation, nucleic acid sorting, and nucleic acid maintenance. Most of these functions involve protein complexes that bind to specific sites in the genome and in RNA sequences. Transcriptional activators, for example, bind to specific promoter sequences and recruit chromatin-modifying complexes to initiate RNA transcription. Distinct DNA binding proteins bind origins of replication, centromeres, telomeres, or other sites in the genome, to regulate DNA replication, condensation, and other aspects of genome maintenance. Specific proteins bind to RNA translation initiation sites, or poly A sites, control translation, RNA stability, and other aspect of RNA function. In case of transcription, protein complexes that bind specific promoters, activators and inhibitors of such interaction can serve as important tools to modulate gene expression. Different methods have been developed to study these interactions, including DNA gel shift assays, DNA-protein complex immuno-precipitation analysis, and DNA or protein microarrays to examine DNA-protein interactions.

Genetic regulatory sequences, mainly promoters, direct transcription of genes, including disease related genes. Therefore, there is a need to identify molecules that modulate gene transcription. In cancer, tumor suppressor genes, such as p53, are usually mutated or disregulated, and other genes, such as tyrosine kinase receptors, are overexpressed (Bruce et al., 1998. Proc Natl Acad Sci U S A. 95:15158-15160; Masahiro et al., 2001. Proc Natl Acad Sci U S A. 98:136-141; Melinda et al., 2000. Proc Natl Acad Sci U S A. 97:5504-5509). The p53 tumor suppressor gene is the most frequently mutated gene present in human cancers. The function of p53 protein is to maintain genetic stability by inducing cell cycle arrest in late GI phase of the cell cycle, and/or apoptosis in response to genotoxic stress (Gottifredi et al., 2001. Proc Natl Acad Sci U S A. 98:1036-1041; Sugrue et al., 1997. ProcNatl Acad Sci U S A. 94:9648-9653; Sjstrom and Bergh, 2001. BMJ 322:1538-1549; Levin, 1997. Cell 88:323-331). The biological effects of p53 are controlled by p53-dependent transactivation via p53 regulatory elements that regulate the expression of downstream target genes of p53, such as APAF-1, a gene whose transcription is induced by p53. Thus, compounds which regulate p53 transcription, can be useful to treat diseases associated with p53 dysfunction, such as cancer.

The present inventors have devised a method to identify lead peptide regulators for activation of p53 transcription, as described below.

Identification and Isolation of Lead p53 Promoter-binding Polypeptides:

A cDNA phage display library for display of cellular proteins is generated using pCC11, as described above, from cultured normal cells following 18 hours of culture under conditions of serum starvation. Serum-starvation synchronizes the cell cycles of the cells by arrest thereof in late G1. Thus, the phage display library comprises cDNAs encoding proteins involved in mediating p53-dependent cell cycle arrest.

DNA sequences of the p53 promoter are amplified by PCR using p53 promoter specific biotinylated primers designed to amplify full-length p53 promoter, and the amplified fragment is incubated in PBS containing phage cDNA library for 1 hour at 37° C. to allow specific association of p53 promoter and phage displayed polypeptides, and specifically bound phage-DNA complexes are isolated using streptavidin-conjugated magnetic beads, using the King Fisher apparatus (Labsystems, Finland). Specifically associated phage-DNA complexes are isolated with streptavidin-conjugated magnetic beads using the King Fisher apparatus (Labsystems, Finland), and used to infect TG1 bacteria to propagate the population of selected phages. The selection is performed three times to enrich the phage population that specifically binds the p53 promoter.

The capacity of selected phages to specifically bind the promoter is verified as follows. Samples of 500 individual phages are spotted onto PVDF membranes, and the spotted membranes are reacted with biotinylated p53 promoter DNA fragment. Detection of phages that specifically bind the DNA fragment is performed by Western immunoblotting assay using streptavidin-conjugated HRP, and a developing assay using a fluorescent HRP substrate. Cellular proteins capable of specifically binding p53 promoter are identified by sequencing phage-displayed cDNA.

Results:

Polypeptides capable of specifically binding p53 promoter or VEGF promoter are identified.

Conclusions:

The present method can be used to identify and isolate lead regulator polypeptides for regulation of p53 or VEGF transcription. Polypeptides capable of regulating p53 or VEGF transcription can be used to treat diseases associated with p53 or VEGF deregulation, such as cancer.

### Example 6

### Identification and Isolation of Polypeptides Capable of Regulating VEGF Gene Transcription

Many disease states, such as cancer, are associated with deregulation of gene expression. For example, many diseases, such as diseases characterized by IGF-I receptor-activated signaling pathways, are associated with VEGF overexpression. One potent approach to treat such diseases would be to employ compounds capable of regulating expression of genes, such as VEGF. To date, however, no satisfactory methods of identifying and isolating compounds capable of regulating transcription of genes exist. In order to fulfill this important need, the present inventors have devised methods of identifying and isolating such compounds, as follows.

Materials and Methods:

A 3.4 kb DNA fragment comprising the VEGF promoter is PCR amplified using the primers shown in Table 1, and the amplified fragment is isolated and

TABLE 1

Oligonucleotide primers for PCR
amplification of VEGF promoter sequences

| Primer name/ | |
|---|---|
| 5′ nucleotide | |
| position* | Primer sequence |
| 5′F-VEGF | 5′-CTGTGCCCTCACTCCCCTGGATCCCTGGG-3′ |
| promoter/−3400 | (SEQ ID NO: 1) |
| 3′R-VEGF | 5′-GGTTTCGGAGGGCCCGACCGGGGCCGGCGC-3′ |
| promoter/−26 | (SEQ ID NO: 2) |

*VEGF gene numbering

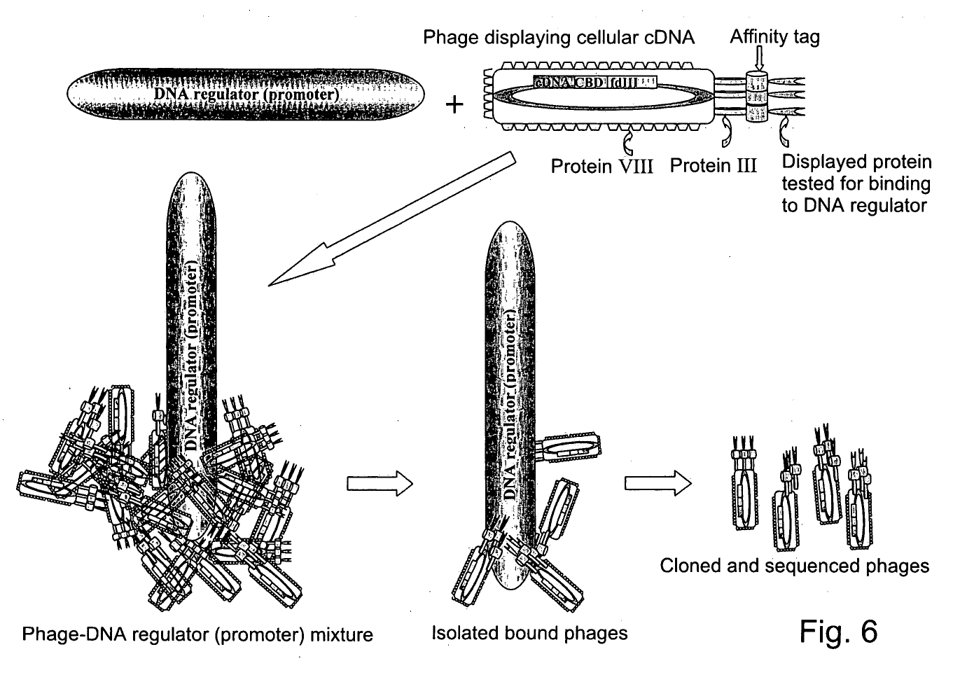

The protocol used to identify and isolate VEGF promoter binding proteins is schematically described in FIG. 6. The tagged VEGF promoter is incubated for one hour at room temperature with an aliquot of cDNA phage display library generated from the IGF-I receptor-expressing human breast cancer cell line T47D to allow formation of specifically bound promoter-phage complexes. The phage display library is generated by cloning cDNA fragments in pCC11, as described above. Specifically associated phage-DNA complexes are isolated with streptavidin-conjugated magnetic beads using the King Fisher apparatus (Labsystems, Finland), and used to infect TG1 bacteria to propagate the population of selected phages. The selection is performed three times to enrich the phage population that specifically binds the VEGF promoter. tagged with biotin.

The capacity of selected phages to specifically bind the promoter is verified as follows. Samples of 500 individual phages are spotted onto PVDF membranes, and the spotted membranes are reacted with biotinylated VEGF promoter DNA fragment. Detection of phages that specifically bind the DNA fragment is performed by Western immunoblotting assay using streptavidin-conjugated HRP, and a developing assay using a fluorescent HRP substrate. Cellular proteins capable of specifically binding VEGF promoter are identified by sequencing phage-displayed cDNA.

Results:

Polypeptides capable of specifically binding the VEGF promoter are identified.

Conclusions:

The present method can be used to identify and isolate lead regulator polypeptides for regulation of VEGF transcription. Polypeptides capable of regulating VEGF transcription constitute potent therapeutic agents which can be used to treat diseases associated with VEGF deregulation, such as cancer.

### Example 7

### Identification of Putative Functional Analogs of Molecular Regulators

Polypeptidic molecular regulators of biological pathways can be relatively easily identified since polypeptides are natural regulators of biological pathways, and since polypeptides are amenable to facile manipulation and functional selection using powerful molecular biological and biochemical methods. The highly specific functionalities of such polypeptide regulators of biological pathways are uniquely useful, for example for pharmaceutical applications. However, polypeptides present numerous drawbacks as pharmaceutical agents. For example, polypeptides do not exhibit optimal physiological stability, are often too large to function optimally as therapeutic agents, or display unacceptable toxicity. Thus, methods of generating regulatory analogs of polypeptide regulators of biological pathways having desired physico-chemical characteristics is highly desirable. However, to date, no satisfactory methods of generating such regulatory analogs exist. In order to fulfill this important need, the present inventors have uncovered methods of generating regulatory analogs of polypeptide regulators of biological pathways, as follows.

Materials and Methods:

Lead functional analogs of peptide regulators of signaling pathways are identified by testing the ability of compounds to inhibit association of peptide regulators and their target ligands. Such compounds are lead compounds for having similar signaling pathway regulatory capacities, or similar target ligand binding specificities as such lead peptide regulators.

First Approach—Substrate-immobilization of Phages Displaying Target Ligands of Lead Peptide Regulators:

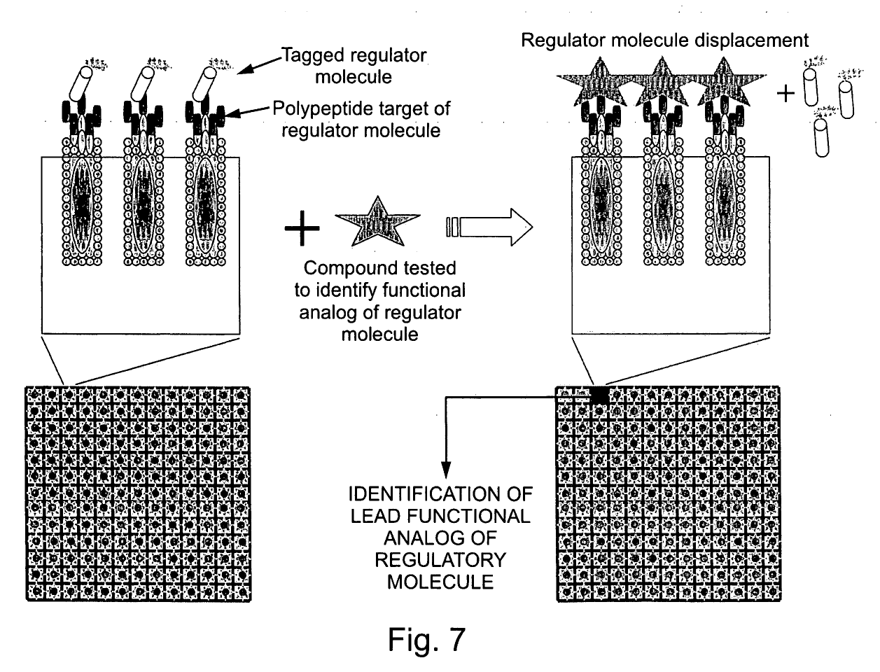

An affinity-tagged (biotin) constituent of a signaling pathway (VEGF promoter or EHD-1) being a target ligand of a lead peptide regulator of the signaling pathway (IGF-I receptor signaling) is attached to a substrate (multi-well plates) to which an affinity tag ligand (streptavidin) has been conjugated. Selected phages displaying the lead peptide regulator fused to a detectable tag (CBD) in PBS are added to the wells, and the plates are incubated for one hour at room temperature solution to allow formation of phage-target ligand complexes. The plates are washed with PBS containing 0.02% Tween to remove non-complexed molecules. Libraries of compounds (non-polypeptidic compounds or compounds being smaller or lighter than the lead peptide regulator) are added to the wells, and the plates are incubated for 30 minutes at room temperature in order to allow displacement of phages from phage-target ligand complexes by the compounds. Aliquots of 50 μl of compounds at a concentration of 100 μg/ml in PBS are added to the plates. The wells are washed with PBS to remove non-adherent molecules, and association of phages with target ligands is measured by adding HRP-conjugated anti phage protein VIII antibodies to the plates and performing an ELISA using a chromogenic HRP substrate. The amount of HRP activity is inversely correlated to the capacity of the compound to inhibit association of phages with target ligands. FIG. 7 demonstrates the screening procedure of such displacement in a high throughput set-up.

Second Approach—Substrate-immobilization of Selected cDNA Phages Displaying Target Ligands of Lead Peptide Regulators:

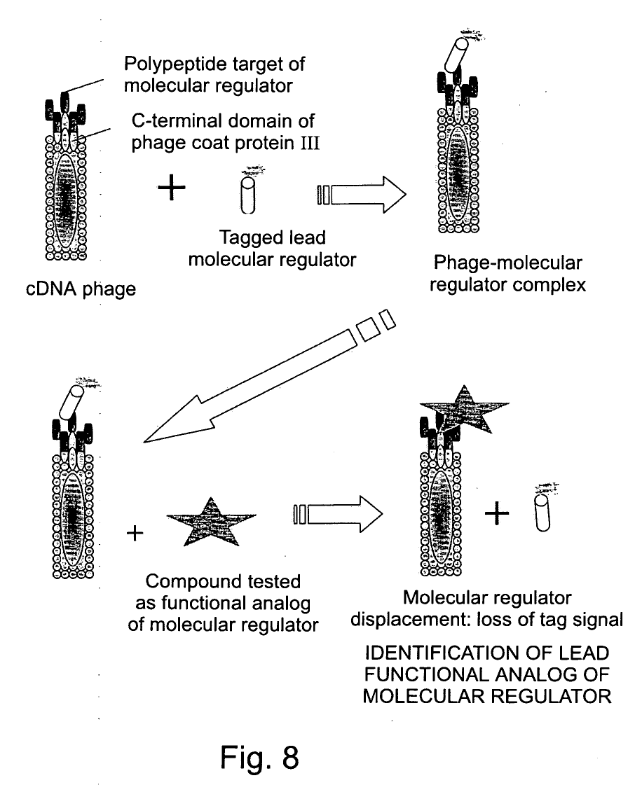

The protocol used for identification of lead functional analogs of using the phage substrate-immobilization approach is depicted in FIG. 8. Selected cDNA phages displaying a lead peptide regulator fused top hage viral coat protein III via an affinity tag are attached to multi-well plate surfaces coated with a phage immobilization ligand. The affinity tag used is CBD, and the immobilization ligand used is anti phage protein pVIII antibodies or a cellulose coated matrix (Berdichevsky et al., 1999. J Immunol Methods 228:151-162). A chimeric polypeptide comprising a detection tag (blue fluorescent protein) and a target ligand bound by lead peptide regulator is added to the wells, and the plates are incubated for one hour at room temperature in PBS solution to allow formation of phage-target ligand complexes. The plates are washed with PBS containing 0.02% Tween to remove non-complexed molecules. Libraries of compounds (non-polypeptidic compounds or compounds being smaller or lighter than the lead peptide regulator) are added to the wells, and the plates are incubated for 10-30 minutes at room temperature in order to allow displacement of target ligands from phage-target ligand complexes by the compounds. Aliquots of 50 μl of compounds at a concentration of 100 μg/ml in PBS are added to the plates. The wells are washed with PBS to remove non-adherent molecules, and association of phages with target ligands is measured by adding HRP-conjugated anti blue fluorescent protein antibodies to the plates and performing an ELISA using a chromogenic HRP substrate. The amount of HRP activity is inversely correlated to the capacity of the compound to inhibit association of phages with target ligands.

Alternately, the compound is conjugated to a detectable tag (FITC), and association of phage-compound complexes is proportional to FITC signal detection after washing.

Coating Plates:

Standard 96 Micro-Well plates (Nunc) specially designed for use in automated equipment with straight sides and deep-skirted lids to offer ample space for reliable gripping of the plates by robotic arms as well as affixing barcodes are used. For binding CBD-tagged phages, Nunc Silent Screen Plates containing cellulose membranes enabling filtration of unbound materials are used. Such plates have membranes which may be peeled from plate following filtration to allow further analysis. Such membranes allow incubation, filtration, immobilization, precipitation and filtrate collection. Aliquots of 50 μl of phage-lead peptide regulator complex solution are added to the wells and the supernatant is filtered out after a 10 minute incubation at room temperature.

Enzyme-linked Immunosorbent Assays (ELISA):

Plates are incubated with blocking solution (2% skim milk powder in PBS) for 30 minutes. A 100 μl aliquot of a 1:5000 dilution of HRP-conjugated anti blue fluorescent protein antibodies or anti phage protein VIII antibodies are added to each well. The plates are incubated for an additional 30 minutes, followed by 3 washes with PBS to remove unbound antibody. For detection of horseradish peroxidase activity, 100 μl of ABTS peroxidase substrate solution (Pharmacia, Uppsala, Sweden) is added to each well, and absorbance is recorded at 405 nm.

Optimization of Lead Functional Analog Compounds:

Selected lead functional analog compounds displaying a desired activity are modified to exhibit optimal activity in-vitro and in-vivo by applying a variety of changes the lead functional analog compound. Modified compounds are re-tested for their ability to inhibit association of target ligands of lead peptide regulators with phages displaying such lead peptide regulators. Optimal lead functional analog compounds suitable for drug development are selected optimally inhibiting association of target ligands of lead peptide regulators with phages displaying such lead peptide regulators, displaying optimal stability under physiological conditions, displaying optimal specificity for the target ligand, and displaying minimal side effects in-vivo.

Results:

Compounds having a similar binding affinity and/or specificity for a signaling pathway target ligand as a peptide regulator of a signaling pathway, but being smaller and/or or lighter than the lead peptide regulator, or being non-polypeptidic, are identified. Lead functional analog compounds displaying a similar regulatory activity as lead peptide regulator compounds are identified and optimized with respect to such regulatory activity.

Conclusions:

The above-described method enables identification and isolation of compounds which can be used as reagents having the same binding specificities and/or regulatory capacities as lead peptide regulators of biological pathways. The method enables identification and isolation of such compounds with s ignificantly greater efficiency than prior art methods. Such compounds, being smaller and/or lighter than lead peptide regulators, or being non-polypeptidic, are optimal for a variety of uses, in particular for use as drugs. Thus, the method of the present invention is superior to all prior art methods of identifying functional analogs of peptide regulators of signaling pathways optimal for use in pharmaceutical applications.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of uncovering a putative functional analog of a peptide regulator of a biological pathway, the method comprising:
(a) generating a library including molecules representing:
(i) constituents of the biological pathway; and/or
(ii) portions of said constituents of the biological pathway;
(b) contacting said molecules of said library with the peptide regulator to thereby obtain a complex composed of a molecule of said molecules of said library and the peptide regulator;
(c) incubating said molecule and the peptide regulator of said complex in the presence of each of a plurality of distinct substances; and
(d) identifying a substance of said plurality of distinct substances capable of competing with the peptide regulator for binding of said molecule to thereby uncover the putative functional analog of the peptide regulator of the biological pathway.

2. The method of claim 1, wherein the peptide regulator comprises a detectable tag, and whereas step (d) is effected by detecting dissociation of said detectable tag from said molecule of said molecules of said library.

3. The method of claim 2, wherein said detectable tag is selected from the group consisting of a fluorescent tag, an enzyme tag, an epitope tag, and an affinity tag.

4. The method of claim 3, wherein said fluorescent tag is selected from the group consisting of green fluorescent protein, blue fluorescent protein, FITC and rhodamine.

5. The method of claim 3, wherein said enzyme is selected from the group consisting of beta-galactosidase, horseradish peroxidase and alkaline phosphatase.

6. The method of claim 3, wherein said affinity tag is selected from the group consisting of a poly-histidine tag, a cellulose binding domain, biotin, avidin, streptavidin, and a DNA-binding domain.

7. The method of claim 1, wherein said molecules of said library comprise a detectable tag, and whereas step (d) is effected by detecting dissociation of said detectable tag from the peptide regulator of said complex.

8. The method of claim 7, wherein said detectable tag is selected from the group consisting of a fluorescent tag, an enzyme tag, an epitope tag, and an affinity tag.

9. The method of claim 8, wherein said fluorescent tag is green fluorescent protein or blue fluorescent protein.

10. The method of claim 8, wherein said enzyme is selected from the group consisting of beta-galactosidase, horseradish peroxidase and alkaline phosphatase.

11. The method of claim 8, wherein said affinity tag is selected from the group consisting of a poly-histidine tag, a cellulose binding domain, biotin, avidin, streptavidin, and a DNA-binding domain.

12. The method of claim 1, wherein said each of a plurality of distinct substances comprises a detectable tag, and whereas step (d) is effected by detecting association of said detectable tag with said molecule of said molecules of said library.

13. The method of claim 12, wherein said detectable tag is selected from the group consisting of a fluorescent tag, an enzyme tag, an epitope tag, and an affinity tag.

14. The method of claim 13, wherein said fluorescent tag is selected from the group consisting of green fluorescent protein, blue fluorescent protein, FITC and rhodamine.

15. The method of claim 13, wherein said enzyme is selected from the group consisting of beta-galactosidase, horseradish peroxidase and alkaline phosphatase.

16. The method of claim 13, wherein said affinity tag is selected from the group consisting of a poly-histidine tag, a cellulose binding domain, biotin, avidin, streptavidin, and a DNA-binding domain.

17. The method of claim 1, wherein the plurality of distinct substances is a plurality of non polypeptide molecules.

18. The method of claim 1, wherein the plurality of distinct substances is a plurality of molecules each having a lower molecular weight than that of the peptide regulator.

19. The method of claim 1, wherein the plurality of distinct substances is a plurality of molecules each having a volume smaller than that of the peptide regulator.

20. The method of claim 1, wherein said library is a display library.

21. The method of claim 20, wherein said display library is a cDNA display library.

22. The method of claim 1, wherein step (a) further comprises fragmenting a pool of polynucleotides by treatment with DNase, or by treatment with a restriction enzyme cleaving at a recognition sequence comprising a number of base pairs less than a range selected from 3 to 7 base pairs, thereby generating a population of polynucleotides encoding said molecules of said library.

23. The method of claim 22, wherein said restriction enzyme is Rsa I or EcoR V.

24. The method of claim 20, wherein said display library is a phage display library.

25. The method of claim 24, wherein said phage display library is a phage display library of polypeptides.

26. The method of claim 25, wherein said polypeptides are composed of a number of amino acid residues less than a range selected from 3 to 34 amino acid residues.

27. The method of claim 25, wherein said polypeptides comprise at least portions of signaling intermediates of the biological pathway.

28. The method of claim 1, wherein said library is prepared from cells containing said constituents of the biological pathway.

29. The method of claim 28, wherein said molecules are polypeptides and whereas said cells are induced to express said polypeptides.

30. The method of claim 29, wherein the biological pathway is associated with regulation of apoptosis and whereas said inducing is effected by treatment with Taxol and/or doxorubicin.

31. The method of claim 29, wherein the biological pathway is an IGF-I receptor activated biological pathway and whereas said inducing is effected by treatment with IGF.

32. The method of claim 1, wherein said library is a cDNA subtraction library constructed to encode polypeptides unique to cells expressing the biological pathway.

33. The method of claim 32, wherein said cDNA subtraction library is derived from a subtraction between a cDNA library generated from cells of a tissue type having a normal phenotype and a cDNA library generated from cells of said tissue type having an abnormal phenotype.

34. The method of claim 33, wherein said tissue type is pulmonary.

35. The method of claim 33, wherein said abnormal phenotype is a cancerous phenotype or a transformed phenotype.

36. The method of claim 1, wherein said library is a cDNA subtraction library constructed to encode polypeptides not present in cells expressing the biological pathway.

37. The method of claim 36, wherein said cDNA subtraction library is derived from a subtraction between a cDNA library generated from cells of a tissue type having a normal phenotype and a cDNA library generated from cells of said tissue type having an abnormal phenotype.

38. The method of claim 37, wherein said tissue type is pulmonary.

39. The method of claim 37, wherein said abnormal phenotype is a cancerous phenotype and/or a transformed phenotype.

40. The method of claim 1, wherein said molecules of said library are signaling intermediates of the biological pathway.

41. The method of claim 40, wherein said signaling intermediates are selected from the group consisting of IRS-1, EHD-1, IGF-I receptor, p53, a vascular growth factor promoter, and an apoptotic protease activating factor-1 promoter.

42. The method of claim 1, wherein said molecules of said library include polypeptides and/or polynucleotides.

43. The method of claim 42, wherein said polynucleotides include gene regulatory elements.

44. The method of claim 43, wherein said gene regulatory elements include promoters.

45. The method of claim 44, wherein said promoters include vascular endothelial growth factor promoters or apoptotic protease activating factor-1 promoters.

46. The method of claim 1, wherein the biological pathway is associated with an abnormal cellular phenotype.

47. The method of claim 46, wherein said abnormal cellular phenotype is a cancerous phenotype and/or an apoptosis resistant phenotype.

48. The method of claim 1, wherein the biological pathway is an IGF-I receptor activated biological pathway.

49. The method of claim 48, wherein said library is prepared from cells selected from the group consisting of NIH 3T3 cells expressing IGF-I receptor, breast cancer cells, placental cells, NIH L1 cells, and adipocytes.

50. The method of claim 49, wherein said breast cancer cells are primary breast cancer cells or cells of a breast cancer cell line.

51. The method of claim 50, wherein said breast cancer cell line is T47D or MCF7.

52. The method of claim 1, wherein the biological pathway is a biological pathway associated with regulation of apoptosis.

53. The method of claim 52, wherein said regulation of apoptosis is activation of apoptosis or inhibition of apoptosis.

54. The method of claim 52, wherein said library is prepared from lung cancer cells.

55. The method of claim 54, wherein said lung cancer cells are primary cancer cells or cells of a lung cancer cell line.

56. The method of claim 54, wherein said lung cancer cells are non small-cell lung cancer cells.

57. The method of claim 55, wherein said cancer cell line is selected from the group consisting of H1299, H522, and H23.

58. The method of claim 1, wherein the biological pathway is a bacterial biological pathway.

59. The method of claim 58, wherein said bacteria is Staphylococcus aureus.

60. A method of uncovering a putative functional analog of a molecular regulator of a biological pathway, the method comprising:
(a) generating a library including molecules representing:
(i) constituents of the biological pathway; and/or
(ii) portions of said constituents of the biological pathway;
(b) contacting said molecules of said library with the molecular regulator to thereby obtain a complex composed of a molecule of said molecules of said library and the molecular regulator;
(c) incubating said molecule and the molecular regulator of said complex in the presence of each of a plurality of distinct substances; and
(d) identifying a substance of said plurality of distinct substances capable of competing with the molecular regulator for binding of said molecule to thereby uncover the putative functional analog of the molecular regulator of the biological pathway.

61. The method of claim 60, wherein said molecular regulator is a molecule selected from the group consisting of a polypeptide, a polynucleotide, a carbohydrate, a biological polymer, and an inorganic molecule.

62. The method of claim 60, wherein said molecular regulator comprises a molecule selected from the group consisting of a polypeptide, a polynucleotide, a carbohydrate, a biological polymer, and an inorganic molecule.

63. The method of claim 60, wherein the molecular regulator comprises a detectable tag, and whereas step (d) is effected by detecting dissociation of said detectable tag from said molecule of said molecules of said library.

64. The method of claim 63, wherein said detectable tag is selected from the group consisting of a fluorescent tag, an enzyme tag, an epitope tag, and an affinity tag.

65. The method of claim 64, wherein said fluorescent tag is selected from the group consisting of green fluorescent protein, blue fluorescent protein, FITC and rhodamine.

66. The method of claim 64, wherein said enzyme is selected from the group consisting of beta-galactosidase, horseradish peroxidase and alkaline phosphatase.

67. The method of claim 64, wherein said affinity tag is selected from the group consisting of a poly-histidine tag, a cellulose binding domain, biotin, avidin, streptavidin, and a DNA-binding domain.

68. The method of claim 60, wherein said molecules of said library comprise a detectable tag, and whereas step (d) is effected by detecting dissociation of said detectable tag from the molecular regulator of said complex.

69. The method of claim 68, wherein said detectable tag is selected from the group consisting of a fluorescent tag, an enzyme tag, an epitope tag, and an affinity tag.

70. The method of claim 69, wherein said fluorescent tag is selected from the group consisting of green fluorescent protein, blue fluorescent protein, FITC and rhodamine.

71. The method of claim 69, wherein said enzyme is selected from the group consisting of beta-galactosidase, horseradish peroxidase and alkaline phosphatase.

72. The method of claim 69, wherein said affinity tag is selected from the group consisting of a poly-histidine tag, a cellulose binding domain, biotin, avidin, streptavidin, and a DNA-binding domain.

73. The method of claim 60, wherein said each of a plurality of distinct substances comprises a detectable tag, and whereas step (d) is effected by detecting association of said detectable tag with said molecule of said molecules of said library.

74. The method of claim 73, wherein said detectable tag is selected from the group consisting of a fluorescent tag, an enzyme tag, an epitope tag, and an affinity tag.

75. The method of claim 74, wherein said fluorescent tag is selected from the group consisting of green fluorescent protein, blue fluorescent protein, FITC and rhodamine.

76. The method of claim 74, wherein said enzyme is selected from the group consisting of beta-galactosidase, horseradish peroxidase and alkaline phosphatase.

77. The method of claim 74, wherein said affinity tag is selected from the group consisting of a poly-histidine tag, a cellulose binding domain, biotin, avidin, streptavidin, and a DNA-binding domain.

78. The method of claim 60, wherein the plurality of distinct substances is a plurality of non polypeptide molecules.

79. The method of claim 60, wherein the plurality of distinct substances is a plurality of molecules each having a lower molecular weight than that of the molecular regulator.

80. The method of claim 60, wherein the plurality of distinct substances is a plurality of molecules each having a volume smaller than that of the molecular regulator.

81. The method of claim 60, wherein said library is a display library.

82. The method of claim 81, wherein said display library is a cDNA display library.

83. The method of claim 60, wherein step (a) further comprises fragmenting a pool of polynucleotides by treatment with DNase, or by treatment with a restriction enzyme cleaving at a recognition sequence comprising a number of base pairs numbering less than a range selected from 3 to 7 base pairs, thereby generating a population of polynucleotides encoding said molecules of said library.

84. The method of claim 83, wherein said restriction enzyme is Rsa I or EcoR V.

85. The method of claim 81, wherein said display library is a phage display library.

86. The method of claim 85, wherein said phage display library is a phage display library of polypeptides.

87. The method of claim 86, wherein said polypeptides are composed of a number of amino acid residues less than a range selected from 3 to 34 amino acid residues.

88. The method of claim 86, wherein said polypeptides comprise at least portions of signaling intermediates of the biological pathway.

89. The method of claim 60, wherein said library is prepared from cells containing said constituents of the biological pathway.

90. The method of claim 89, wherein said molecules are polypeptides and whereas said cells are induced to express said polypeptides.

91. The method of claim 90, wherein the biological pathway is associated with regulation of apoptosis and whereas said inducing is effected by treatment with Taxol and/or doxorubicin.

92. The method of claim 90, wherein the biological pathway is an IGF-I receptor activated biological pathway and whereas said inducing is effected by treatment with IGF.

93. The method of claim 60, wherein said library is a cDNA subtraction library constructed to encode polypeptides unique to cells expressing the biological pathway.

94. The method of claim 93, wherein said cDNA subtraction library is derived from a subtraction between a cDNA library generated from cells of a tissue type having a normal phenotype and a cDNA library generated from cells of said tissue type having an abnormal phenotype.

95. The method of claim 94, wherein said tissue type is pulmonary.

96. The method of claim 94, wherein said abnormal phenotype is a cancerous phenotype or a transformed phenotype.

97. The method of claim 60, wherein said library is a cDNA subtraction library constructed to encode polypeptides not present in cells expressing the biological pathway.

98. The method of claim 97, wherein said cDNA subtraction library is derived from a subtraction between a cDNA library generated from cells of a tissue type having a normal phenotype and a cDNA library generated from cells of said tissue type having an abnormal phenotype.

99. The method of claim 98, wherein said tissue type is pulmonary.

100. The method of claim 98, wherein said abnormal phenotype is a cancerous phenotype and/or a transformed phenotype.

101. The method of claim 60, wherein said molecules of said library are signaling intermediates of the biological pathway.

102. The method of claim 101, wherein said signaling intermediates are selected from the group consisting of IRS-1, EHD-1, IGF-I receptor, p53, a vascular growth factor promoter, and an apoptotic protease activating factor-1 promoter.

103. The method of claim 60, wherein said molecules of said library include polypeptides and/or polynucleotides.

104. The method of claim 103, wherein said polynucleotides include gene regulatory elements.

105. The method of claim 104, wherein said gene regulatory elements include promoters.

106. The method of claim 105, wherein said promoters include vascular endothelial growth factor promoters or apoptotic protease activating factor-1 promoters.

107. The method of claim 60, wherein the biological pathway is associated with an abnormal cellular phenotype.

108. The method of claim 107, wherein said abnormal cellular phenotype is a cancerous phenotype and/or an apoptosis resistant phenotype.

109. The method of claim 60, wherein the biological pathway is an IGF-I receptor activated biological pathway.

110. The method of claim 109, wherein said library is prepared from cells selected from the group consisting of NIH 3T3 cells expressing IGF-I receptor, breast cancer cells, placental cells, NIH L1 cells, and adipocytes.

111. The method of claim 110, wherein said breast cancer cells are primary breast cancer cells or cells of a breast cancer cell line.

112. The method of claim 111, wherein said breast cancer cell line is T47D or MCF7.

113. The method of claim 60, wherein the biological pathway is a biological pathway associated with regulation of apoptosis.

114. The method of claim 113, wherein said regulation of apoptosis is activation of apoptosis or inhibition of apoptosis.

115. The method of claim 113, wherein said library is prepared from lung cancer cells.

116. The method of claim 115, wherein said lung cancer cells are primary cancer cells or cells of a lung cancer cell line.

117. The method of claim 115, wherein said lung cancer cells are non small-cell lung cancer cells.

118. The method of claim 116, wherein said cancer cell line is selected from the group consisting of H1299, H522, and H23.

119. The method of claim 60, wherein the biological pathway is a bacterial biological pathway.

120. The method of claim 119, wherein said bacteria is Staphylococcus aureus.

121. A method of characterizing a molecular target of a peptide regulator of a biological pathway, the method comprising:
(a) generating a library including molecules representing:
(i) constituents of the biological pathway; and/or
(ii) portions of said constituents of the biological pathway; and
(b) screening said molecules of said library for a molecule capable of specifically binding the peptide regulator of the biological pathway, thereby characterizing the molecular target of the peptide regulator.

122. The method of claim 121, wherein, said screening said library comprises:
(i) attaching the peptide regulator to a substrate;
(ii) exposing the peptide regulator to said molecules of said library, to thereby obtain complexes each composed of the peptide regulator and a molecule of said molecules; and
(iii) isolating said complexes.

123. The method of claim 121, further comprising identifying said molecule of said complexes isolated in step (iii).

124. The method of claim 121, wherein said library is a display library.

125. The method of claim 124, wherein said display library is a cDNA display library.

126. The method of claim 121, wherein step (a) further comprises fragmenting a pool of polynucleotides comprising nucleic acid sequences encoding said molecules of said library by treatment with DNase, or by treatment with a restriction enzyme cleaving at a recognition sequence comprising a number of base pairs numbering less than a range selected from 3 to 7 base pairs, thereby generating a population of polynucleotides encoding said molecules of said library.

127. The method of claim 126, wherein said restriction enzyme is Rsa I or EcoR V.

128. The method of claim 124, wherein said display library is a phage display library.

129. The method of claim 128, wherein said phage display library is a phage display library of polypeptides.

130. The method of claim 129, wherein said polypeptides are composed of a number of amino acid residues less than a range selected from 3 to 34 amino acid residues.

131. The method of claim 129, wherein said polypeptides comprise at least portions of signaling intermediates of the biological pathway.

132. The method of claim 121, wherein said library is prepared from cells containing said constituents of the biological pathway.

133. The method of claim 132, wherein said molecules are polypeptides and whereas said cells are induced to express said polypeptides.

134. The method of claim 133, wherein the biological pathway is associated with regulation of apoptosis and whereas said inducing is effected by treatment with Taxol and/or doxorubicin.

135. The method of claim 133, wherein the biological pathway is an IGF-I receptor activated biological pathway and whereas said inducing is effected by treatment with IGF.

136. The method of claim 121, wherein said library is a cDNA subtraction library constructed to encode polypeptides unique to cells expressing the biological pathway.

137. The method of claim 136, wherein said cDNA subtraction library is derived from a subtraction between a cDNA library generated from cells of a tissue type having a normal phenotype and a cDNA library generated from cells of said tissue type having an abnormal phenotype.

138. The method of claim 137, wherein said tissue type is pulmonary.

139. The method of claim 137, wherein said abnormal phenotype is a cancerous phenotype or a transformed phenotype.

140. The method of claim 121, wherein said library is a cDNA subtraction library constructed to encode polypeptides not present in cells expressing the biological pathway.

141. The method of claim 140, wherein said cDNA subtraction library is derived from a subtraction between a cDNA library generated from cells of a tissue type having a normal phenotype and a cDNA library generated from cells of said tissue type having an abnormal phenotype.

142. The method of claim 141, wherein said tissue type is pulmonary.

143. The method of claim 141, wherein said abnormal phenotype is a cancerous phenotype or a transformed phenotype.

144. The method of claim 121, wherein said molecules of said library are signaling intermediates of the biological pathway.

145. The method of claim 144, wherein said signaling intermediates are selected from the group consisting of IRS-1, EHD-1, IGF-I receptor, p53, a vascular growth factor promoter, and an apoptotic protease activating factor-1 promoter.

146. The method of claim 121, wherein said molecules of said library include polypeptides and/or polynucleotides.

147. The method of claim 146, wherein said polynucleotides include gene regulatory elements.

148. The method of claim 147, wherein said gene regulatory elements include promoters.

149. The method of claim 148, wherein said promoters include vascular endothelial growth factor promoters or apoptotic protease activating factor-1 promoters.

150. The method of claim 121, wherein the biological pathway is associated with an abnormal cellular phenotype.

151. The method of claim 150, wherein said abnormal cellular phenotype is a cancerous phenotype and/or an apoptosis resistant phenotype.

152. The method of claim 121, wherein the biological pathway is an IGF-I receptor activated biological pathway.

153. The method of claim 152, wherein said library is prepared from cells selected from the group consisting of NIH 3T3 cells expressing IGF-I receptor, breast cancer cells, placental cells, NIH L1 cells, and adipocytes.

154. The method of claim 153, wherein said breast cancer cells are primary breast cancer cells or cells of a breast cancer cell line.

155. The method of claim 154, wherein said breast cancer cell line is T47D or MCF7.

156. The method of claim 121, wherein the biological pathway is a biological pathway associated with regulation of apoptosis.

157. The method of claim 156, wherein said regulation of apoptosis is activation of apoptosis or inhibition of apoptosis.

158. The method of claim 156, wherein said library is prepared from lung cancer cells.

159. The method of claim 158, wherein said lung cancer cells are primary cancer cells or cells of a lung cancer cell line.

160. The method of claim 158, wherein said lung cancer cells are non small-cell lung cancer cells.

161. The method of claim 159, wherein said cancer cell line is selected from the group consisting of H1299, H522, and H23.

162. The method of claim 121, wherein the biological pathway is a bacterial biological pathway.

163. The method of claim 162, wherein said bacterial biological pathway is a Staphylococcus aureus biological pathway.

164. A method of characterizing a molecular target of a molecular regulator of a biological pathway, the method comprising:
(a) generating a library including molecules representing:
(i) constituents of the biological pathway; and/or
(ii) portions of said constituents of the biological pathway; and
(b) screening said molecules of said library for a molecule capable of specifically binding the molecular regulator of the biological pathway, thereby characterizing the molecular target of the molecular regulator.

165. The method of claim 164, wherein, said screening said library comprises:
(i) attaching the molecular regulator to a substrate;
(ii) exposing the molecular regulator to said molecules of said library, to thereby obtain complexes each composed of the molecular regulator and a molecule of said molecules; and
(iii) isolating said complexes.

166. The method of claim 164, further comprising identifying said molecule of said complexes isolated in step (iii).

167. The method of claim 164, wherein the molecular regulator is a polynucleotide.

168. The method of claim 167, wherein said polynucleotide includes a gene regulatory element.

169. The method of claim 167, wherein said gene regulatory element is a promoter.

170. The method of claim 169, wherein said promoter is a vascular endothelial growth factor promoter or an apoptotic protease activating factor-1 promoter.

171. The method of claim 164, wherein said library is a display library.

172. The method of claim 171, wherein said display library is a cDNA display library.

173. The method of claim 164, wherein step (a) further comprises fragmenting a pool of polynucleotides by treatment with DNase, or by treatment with a restriction enzyme cleaving at a recognition sequence comprising a number of base pairs numbering less than a range selected from 3 to 7 base pairs, thereby generating a population of polynucleotides encoding said molecules of said library.

174. The method of claim 173, wherein said restriction enzyme is Rsa I or EcoR V.

175. The method of claim 171, wherein said display library is a phage display library.

176. The method of claim 175, wherein said phage display library is a phage display library of polypeptides.

177. The method of claim 176, wherein said polypeptides are composed of a number of amino acid residues less than a range selected from 3 to 34 amino acid residues.

178. The method of claim 176, wherein said polypeptides comprise at least portions of signaling intermediates of the biological pathway.

179. The method of claim 164, wherein said library is prepared from cells containing said constituents of the biological pathway.

180. The method of claim 179, wherein said molecules are polypeptides and whereas said cells are induced to express said polypeptides.

181. The method of claim 180, wherein the biological pathway is associated with regulation of apoptosis and whereas said inducing is effected by treatment with Taxol and/or doxorubicin.

182. The method of claim 180, wherein the biological pathway is an IGF-I receptor activated biological pathway and whereas said inducing is effected by treatment with IGF.

183. The method of claim 164, wherein said library is a cDNA subtraction library constructed to encode polypeptides unique to cells expressing the biological pathway.

184. The method of claim 183, wherein said cDNA subtraction library is derived from a subtraction between a cDNA library generated from cells of a tissue type having a normal phenotype and a cDNA library generated from cells of said tissue type having an abnormal phenotype.

185. The method of claim 184, wherein said tissue type is pulmonary.

186. The method of claim 184, wherein said abnormal phenotype is a cancerous phenotype or a transformed phenotype.

187. The method of claim 164, wherein said library is a cDNA subtraction library constructed to encode polypeptides not present in cells expressing the biological pathway.

188. The method of claim 187, wherein said cDNA subtraction library is derived from a subtraction between a cDNA library generated from cells of a tissue type having a normal phenotype and a cDNA library generated from cells of said tissue type having an abnormal phenotype.

189. The method of claim 188, wherein said tissue type is pulmonary.

190. The method of claim 188, wherein said abnormal phenotype is a cancerous phenotype or a transformed phenotype.

191. The method of claim 164, wherein said molecules of said library are signaling intermediates of the biological pathway.

192. The method of claim 191, wherein said signaling intermediates are selected from the group consisting of IRS-1, EHD-1, IGF-I receptor, p53, a vascular growth factor promoter, and an apoptotic protease activating factor-1 promoter.

193. The method of claim 164, wherein said molecules of said library include polypeptides and/or polynucleotides.

194. The method of claim 193, wherein said polynucleotides include gene regulatory elements.

195. The method of claim 194, wherein said gene regulatory elements include promoters.

196. The method of claim 195, wherein said promoters include vascular endothelial growth factor promoters or apoptotic protease activating factor-1 promoters.

197. The method of claim 164, wherein the biological pathway is associated with an abnormal cellular phenotype.

198. The method of claim 197, wherein said abnormal cellular phenotype is a cancerous phenotype and/or an apoptosis resistant phenotype.

199. The method of claim 164, wherein the biological pathway is an IGF-I receptor activated biological pathway.

200. The method of claim 199, wherein said library is prepared from cells selected from the group consisting of NIH 3T3 cells expressing IGF-I receptor, breast cancer cells, placental cells, NIH L1 cells, and adipocytes.

201. The method of claim 200, wherein said breast cancer cells are primary breast cancer cells or cells of a breast cancer cell line.

202. The method of claim 201, wherein said breast cancer cell line is T47D or MCF7.

203. The method of claim 164, wherein the biological pathway is a biological pathway associated with regulation of apoptosis.

204. The method of claim 203, wherein said regulation of apoptosis is activation of apoptosis or inhibition of apoptosis.

205. The method of claim 203, wherein said library is prepared from lung cancer cells.

206. The method of claim 205, wherein said lung cancer cells are primary cancer cells or cells of a lung cancer cell line.

207. The method of claim 205, wherein said lung cancer cells are non small-cell lung cancer cells.

208. The method of claim 206, wherein said cancer cell line is selected from the group consisting of H1299, H522, and H23.

209. The method of claim 164, wherein the biological pathway is a bacterial biological pathway.

210. The method of claim 209, wherein said bacterial biological pathway is a Staphylococcus aureus biological pathway.

211. An expression construct system comprising a plurality of expression constructs being for phage display expression of polypeptides, each of said expression constructs having a unique polylinker sequence flanked by:
(a) a first polynucleotide region encoding a phage leader sequence positioned upstream of said polylinker; and
(b) a second polynucleotide region encoding a chimeric polypeptide including a tag sequence fused to a phage coat protein;
wherein each unique polylinker is designed to enable cloning of a desired polynucleotide in a unique reading frame combination with respect to said leader sequence and said chimeric polypeptide, such that phage particles expressing said desired polynucleotide cloned in frame to said leader sequence and said chimeric polypeptide can be identified and optionally isolated from a phage particle population transformed with said plurality of expression constructs harboring said desired polynucleotide.

212. The expression construct system of claim 211, wherein said phage leader sequence is a gene 3 leader sequence.

213. The expression construct system of claim 211, wherein said tag sequence is selected from the group consisting of a fluorescent tag, an enzyme tag, an epitope tag, and an affinity tag.

214. The expression construct system of claim 213, wherein said fluorescent tag is selected from the group consisting of green fluorescent protein or blue fluorescent protein.

215. The expression construct system of claim 213, wherein said enzyme is selected from the group consisting of beta-galactosidase, horseradish peroxidase and alkaline phosphatase.

216. The expression construct system of claim 213, wherein said affinity tag is selected from the group consisting of a poly-histidine tag, a cellulose binding domain, avidin, streptavidin, and a DNA-binding domain.

217. The expression construct system of claim 211, wherein said phage coat protein is coat protein III.

218. The expression construct system of claim 211, wherein said phage particles are M13 phage particles.

219. The expression construct system of claim 211, wherein said desired polynucleotide is a cDNA encoding at least a portion of a constituent of a biological pathway.


SEQUENCE LISTING (20 sequences)

SEQ ID NO: 1 - DNA, 29 bp, Artificial sequence
  Single strand DNA oligonucleotide
ctgtgccctcactcccctggatccctggg

SEQ ID NO: 2 - DNA, 30 bp, Artificial sequence
  Single strand DNA oligonucleotide
ggtttcggagggcccgaccggggccggcgc

SEQ ID NO: 3 - DNA, 72 bp, Artificial sequence
  Polylinker adaptor sense oligonucleotide sequence
catgggggcccggttacggtaccggatccagatatcgaacaaaaactcatctcagaagaagatctgtagggc

SEQ ID NO: 4 - DNA, 72 bp, Artificial sequence
  Polylinker adaptor anti-sense oligonucleotide sequence
ggccgccctacagatcttcttctgagatgagtttttgttcgatatctggatccggtaccgtaaccgggcccc

SEQ ID NO: 5 - DNA, 73 bp, Artificial sequence
  Polylinker adaptor sense oligonucleotide sequence
catgggggcccggttacggtaccggatccagatatccgaacaaaaactcatctcagaagaagatctgtagggc

SEQ ID NO: 6 - DNA, 73 bp, Artificial sequence
  Polylinker adaptor anti-sense oligonucleotide sequence
ggccgccctacagatcttcttctgagatgagtttttgttcggatatctggatccggtaccgtaaccgggcccc

SEQ ID NO: 7 - DNA, 74 bp, Artificial sequence
  Polylinker adaptor sense oligonucleotide sequence
catgggggcccggttacggtaccggatccagatatcccgaacaaaaactcatctcagaagaagatctgtagggc

SEQ ID NO: 8 - DNA, 74 bp, Artificial sequence
  Polylinker adaptor anti-sense oligonucleotide sequence
ggccgccctacagatcttcttctgagatgagtttttgttcgggatatctggatccggtaccgtaaccgggcccc

SEQ ID NO: 9 - DNA, 73 bp, Artificial sequence
  Polylinker adaptor sense oligonucleotide sequence
cgggatgtctagaagaagactctactcaaaaacaagctatagacctaggccatggccattggcccgggggtac

SEQ ID NO: 10 - DNA, 73 bp, Artificial sequence
  Polylinker adaptor anti-sense oligonucleotide sequence
ggccgccctacagatcttcttctgagatgagtttttgttcgatatctggatccggtaccggtaaccgggcccc

SEQ ID NO: 11 - DNA, 74 bp, Artificial sequence
  Polylinker adaptor sense oligonucleotide sequence
catgggggcccggttaccggtaccggatccagatatccgaacaaaaactcatctcagaagaagatctgtagggc

SEQ ID NO: 12 - DNA, 74 bp, Artificial sequence
  Polylinker adaptor anti-sense oligonucleotide sequence
ggccgccctacagatcttcttctgagatgagtttttgttcggatatctggatccggtaccggtaaccgggcccc

SEQ ID NO: 13 - DNA, 75 bp, Artificial sequence
  Polylinker adaptor sense oligonucleotide sequence
catgggggcccggttaccggtaccggatccagatatcccgaacaaaaactcatctcagaagaagatctgtagggc

SEQ ID NO: 14 - DNA, 75 bp, Artificial sequence
  Polylinker adaptor anti-sense oligonucleotide sequence
ggccgccctacagatcttcttctgagatgagtttttgttcgggatatctggatccggtaccggtaaccgggcccc

SEQ ID NO: 15 - DNA, 74 bp, Artificial sequence
  Polylinker adaptor sense oligonucleotide sequence
catgggggcccggttacccggtaccggatccagatatcgaacaaaaactcatctcagaagaagatctgtagggc

SEQ ID NO: 16 - DNA, 74 bp, Artificial sequence
  Polylinker adaptor anti-sense oligonucleotide sequence
ggccgccctacagatcttcttctgagatgagtttttgttcgatatctggatccggtaccgggtaaccgggcccc

SEQ ID NO: 17 - DNA, 75 bp, Artificial sequence
  Polylinker adaptor sense oligonucleotide sequence
catgggggcccggttacccggtaccggatccagatatccgaacaaaaactcatctcagaagaagatctgtagggc

SEQ ID NO: 18 - DNA, 75 bp, Artificial sequence
  Polylinker adaptor anti-sense oligonucleotide sequence
ggccgccctacagatcttcttctgagatgagtttttgttcggatatctggatccggtaccgggtaaccgggcccc

SEQ ID NO: 19 - DNA, 76 bp, Artificial sequence
  Polylinker adaptor sense oligonucleotide sequence
catgggggcccggttacccggtaccggatccagatatcccgaacaaaaactcatctcagaagaagatctgtagggc

SEQ ID NO: 20 - DNA, 76 bp, Artificial sequence
  Polylinker adaptor anti-sense oligonucleotide sequence
ggccgccctacagatcttcttctgagatgagtttttgttcgggatatctggatccggtaccgggtaaccgggcccc